(19) United States
(12) Patent Application Publication
(10) Pub. No.: US 2021/0087159 A1
(43) Pub. Date: Mar. 25, 2021

(54) TETRAHYDROCANNABINOLIC- CANNABINOLIC ACID DERIVATIVES AND USES THEREOF

(71) Applicant: AL&AM PHARMACHEM LTD., Rehovot (IL)

(72) Inventor: Alexander AIZIKOVICH, Rehovot (IL)

(21) Appl. No.: 17/111,432

(22) Filed: Dec. 3, 2020

Related U.S. Application Data
(63) Continuation-in-part of application No. PCT/IL2019/050564, filed on May 19, 2019.
(60) Provisional application No. 62/680,110, filed on Jun. 4, 2018.

(51) Int. Cl. C07D 311/80; C07C 69/94

(52) U.S. Cl. CPC C07D 311/80; C07C 69/94

(57) ABSTRACT

THCA- and CBDA-derivatives can be formulated in pharmaceutical compositions. The derivatives are non-classical cannabinoids that are either agonists or antagonists of the peripheral CB1 and/or CB2 receptors, and are thus useful for CB1 and/or CB2 receptor activation/deactivation, such as for preventing, alleviating, or treating medical conditions that benefit from CB1 and/or CB2 receptor agonist/antagonist treatment.

### CROSS-REFERENCE TO RELATED APPLICATIONS

The present application is a continuation-in-part application of International Application No. PCT/IL2019/050564 filed May 19, 2019, designating the U.S. and published as WO 2019/234728 on Dec. 12, 2019 which claims the benefit of U.S. Provisional Patent Application No. 62/680,110 filed Jun. 4, 2018. Any and all applications for which a foreign or domestic priority claim is identified above and/or in the Application Data Sheet as filed with the present application are hereby incorporated by reference under 37 CFR 1.57.

### BACKGROUND OF THE INVENTION

### Field of the Invention

The present invention provides tetrahydrocannabinolic acid (THCA)- and cannabidiolic acid (CBDA)-derivatives, and pharmaceutical compositions thereof.

Abbreviations: ACN, acetonitrile; CBD, cannabidiol; CBDA, cannabidiolic acid; CDI, carbonyldiimidazole; DCC, dicyclohexylcarbodiimid; DCM, dichloromethane; DMF, dimethylformamide; DMSO, dimethyl sulfoxide; EA, ethyl acetate; HPLC, high performance liquid chromatography; LCMS, liquid chromatography-mass spectrometry; NMR, nuclear magnetic resonance; PE, petroleum ether; THC, Δ⁹-tetrahydrocannabinol; THCA, tetrahydrocannabinolic acid; THF, tetrahydrofuran; TLC, thin layer chromatography.

### Description of the Related Art

Cannabis is known in the history of human civilization as a natural drug for the treatment of various medical conditions such as inflammation, pain, psychoses, migraine and other disorders of nervous system. Owing to their various activity, natural cannabinoids can often be used for the development of new potential drugs especially as starting materials for organic synthesis.

CBDA and THCA are the first isolated products of all types of marihuana, which are then being decarboxylated to their corresponding cannabinoids such as CBD and THC. This process occurs very easily by heating of these acids to a temperature above 100° C., but it can also take place under the influence of light at ordinary temperature on marihuana in natural condition. This fact causes the low stability of THCA and even more so CBDA and, and consequently the complexity of their research and use as potential drugs. At the same time, their high anti-inflammatory (Ruhaak et al., 2011; Grotenhermen et al., 2017), anti-neurodegenerative (Moldzio et al., 2012), and anticancer (Massi et al., 2010; Chakravarti et al., 2014; Pacher, 2013) activities suggest that analogues thereof, which are resistant to heat and light, might be of considerable interest.

U.S. Pat. No. 3,856,820 discloses a 2-aminomethyl dibenzo[b,d]pyran derivative of the formula:

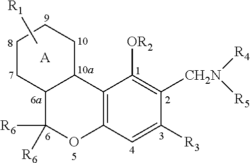

wherein A is a benzene ring, a cyclohexane ring, or a cyclohexene ring with the double bond being at position 6α-10α, 8 or 9; R₁ is H, methyl, or ethyl; R₂ is H or (C₂-C₅)alkanoyl; R₃ is (C₅-C₁₂)alkyl; R₄ and R₅ are H or (C₁-C₄)alkyl, or R₄ and R₅ together with the nitrogen atom to which they are attached form a piperidine or a pyrrolidine ring; and R₆ is methyl or ethyl.

Zehavi and Mechoulam (1981) discloses O- and C-D-glucosyluronic acid derivatives of Δ¹-tetrahydrocannabinol.

WO 2015032519 discloses esters of CBDA with aliphatic alcohols. Similar compounds are disclosed in Crombie et al. (1977), Crombie and Crombie (1977), and Ahmed et al. (2008). Harvey (1977) discloses silyl derivatives and cyclic alkylboronates of CBDA.

Amides of CBDA are of great interest due to their possible therapeutical properties; however, till now only the two specific THCA amides shown herein as compounds 1 and 2 have been described. Compound 1 (3-((6aR,10aR)-1-hydroxy-6,6,9-trimethyl-3-pentyl-6a,7,8,10a-tetrahydro-6H-benzo[c]chromene-2-carboxamido)propanoic acid) was obtained as disclosed in Goto et al. (1994), using two steps synthesis including coupling of THCA with N-hydroxysuccinimide in the presence of DCC with following reaction of pure ester of THCA with 2-aminopropionic acid; and compound 2 ((6aR,10aR)—N-(2,4-dinitrophenyl)-1-hydroxy-6,6,9-trimethyl-3-pentyl-6a,7,8,10a-tetrahydro-6H-benzo[c]chromene-2-carboxamide) was synthesized as disclosed in Gaoni and Mechoulam (1971), by the reaction of THC with 2,4-dinitrobenzoyl azide. No information about the activity of these compounds is provided in said publications. Amido derivatives of CBDA have not been described in the literature up to the present time.

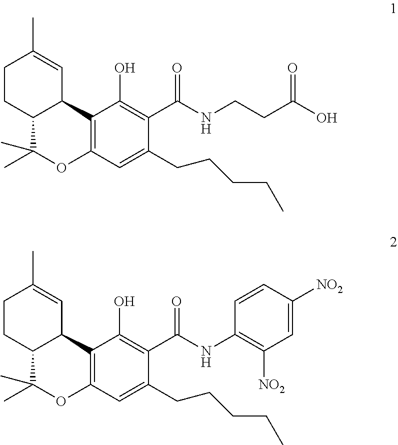 [SMILES: CCCCCc1cc2c(c(O)c1C(=O)NCCC(=O)O)[C@@H]1C=C(C)CC[C@H]1C(C)(C)O2 ; CCCCCc1cc2c(c(O)c1C(=O)Nc1ccc([N+](=O)[O-])cc1[N+](=O)[O-])[C@@H]1C=C(C)CC[C@H]1C(C)(C)O2]

WO 2018/005188 discloses a formulation for inhalation, comprising a THCA- or CBDA mono-, di- or tri-(C₁-C₁₀)alkylamide derivatives.

### SUMMARY OF INVENTION

In one aspect, the present invention provides a compound, more particularly a THCA or CBDA derivative, of the formula I:

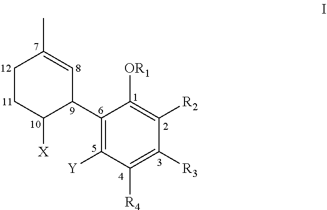

wherein:

X is the radical

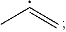

### and Y is —OH or —OR₅; or

X is the diradical

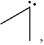

and Y is —O—, and together with X and the carbon atoms to which they are attached form a dihydropyran ring,

or an enantiomer, diastereomer, racemate, or pharmaceutically acceptable salt thereof,

wherein:

R₁ is absent, H, R₅, or —C(O)—;

R₂ is —CN, —C(O)NR₇—, —C(O)N(R₇)₂, —C(O)S—R₇, —C(S)N(R₇)₂, —CH₂N(R₇)₂, or a 5-6-membered aliphatic or aromatic heterocyclyl containing 1-4 heteroatoms and optionally substituted with one or more (C₁-C₄)alkyl groups;

R₃ is —(C₁-C₂)alkyl;

R₄ is H, halogen, —NO, —NO₂, or —NH₂;

R₅ is —(C₁-C₂)alkyl, —(C₁-C₁₂)haloalkyl, —(C₂-C₁₂)alkenyl, —(C₂-C₁₂)alkynyl, —(C₃-C₁₀)cycloalkyl, —(C₁-C₁₂)alkyl-aryl, —(C₁-C₁₂)alkyl-heterocyclyl, —C(O)—R₆, —SiHₙ(R₆)₃₋ₙ, —BHₙ(R₆)₃₋ₙ, —SO₂—R₆;

R₆ is —(C₁-C₁₂)alkyl, —(C₁-C₁₂)haloalkyl, —(C₆-C₁₂)aryl, or —(C₃-C₁₂)heterocyclyl;

R₇ each independently is H, —OR₈, —N(R₈)₂, —N═C(R₈)₂, —NHC(O)R₈, —NHC(S)R₈, a 5-6-membered aliphatic or aromatic heterocyclic ring containing 1-4 heteroatoms, —(C₁-C₁₂)alkyl substituted with one or more groups each independently selected from —OH, —N(R₉)₂, —O(C₁-C₁₂)alkyl, —(C₃-C₁₀)cycloalkyl, and —(C₃-C₁₂)heterocyclyl, or phenyl optionally substituted with one or more groups each independently selected from —OH, —N(R₉)₂, —O(C₁-C₁₂)alkyl, —(C₃-C₁₀)cycloalkyl, and —(C₃-C₂)heterocyclyl, or two R₇ together with the nitrogen atom to which they are attached form a 5- or 6-membered aliphatic or aromatic heterocyclic ring containing 1-4 heteroatoms;

R₈ each independently is H, —(C₁-C₁₂)alkyl, —(C₁-C₁₂)haloalkyl, —(C₃-C₁₀)cycloalkyl, —(C₁-C₁₂)alkyl-aryl, —(C₁-C₁₂)alkyl-heterocyclyl, —(C₆-C₁₂)aryl, —(C₃-C₁₂)heterocyclyl, or —N(R₉)₂;

R₉ each independently is H, —(C₁-C₁₂)alkyl, —(C₁-C₁₂)haloalkyl, —(C₃-C₁₀)cycloalkyl, —(C₁-C₁₂)alkyl-aryl, —(C₁-C₁₂)alkyl-heterocyclyl, —(C₆-C₁₂)aryl, —(C₃-C₁₂)heterocyclyl; and

n is an integer of 0 to 3,

provided that (i) when R₁ is absent or —C(O)—, R₂ is —C(O)NR₇—, and R₁ and R₂ together with the atoms to which they are attached form a 5- or 6-membered heterocyclic ring; (ii) when R₁ is H or R₅, R₂ is not —C(O)NR₇—; and (iii) when R₂ is —CH₂N(R₇)₂, R₇ are not both H.

The compounds of the formula I exemplified herein are identified as compounds Ia₁, Ia₃-Ia₂₅, Ia₃₁-Ia₃₃, Ib₁ and Ic₁ (THCA derivatives) and Ia₂₆-Ia₂₈, and Ia₃₀ (CBDA derivatives), and are shown in Tables 2-3 herein. Additional tetrahydrocannabinolic- and cannabidiolic acid derivatives described are identified herein as compounds Ia₂ and Ia₂₉, respectively.

The THCA and CBDA derivatives of the present invention may be considered as non-classical cannabinoids that are ligands (agonists/antagonists) of the peripheral cannabinoid type 1 (CB1) and/or cannabinoid type 2 (CB2) receptors and are thus expected to be useful for CB1 and/or CB2 receptor activation/deactivation, i.e., for preventing, alleviating, or treating medical conditions that benefit from CB1 and/or CB2 receptor agonist/antagonist treatment. Examples of such medical conditions, without being limited to, include inflammatory diseases, pain or conditions associated therewith, brain or spinal cord diseases, skin diseases, immunological diseases including autoimmune diseases, neurologic diseases, neurodegenerative diseases or disorders, neuroinflammatory conditions, and cancer. As for example shown herein, such compounds are highly capable of inhibiting cancer cell growth.

In another aspect, the present invention thus provides a pharmaceutical composition comprising a compound as defined above, i.e., a THCA or CBDA derivative of the formula I, or an enantiomer, diastereomer, racemate, or pharmaceutically acceptable salt thereof, and a pharmaceutically acceptable carrier. The compositions disclosed herein may be used for CB1 and/or CB2 receptor activation/deactivation, more specifically for treatment of medical conditions that benefit, or may benefit, from CB1 and/or CB2 receptor agonist/antagonist treatment.

In yet another aspect, the present invention thus relates to a compound as defined above, i.e., a THCA or CBDA derivative of the formula I, or an enantiomer, diastereomer, racemate, or pharmaceutically acceptable salt thereof, for use in CB1 and/or CB2 receptor activation/deactivation.

In still another aspect, the present invention relates to use of a compound as defined above, i.e., a THCA or CBDA derivative of the formula I, or an enantiomer, diastereomer, racemate, or pharmaceutically acceptable salt thereof, in the manufacture of a pharmaceutical composition for CB1 and/or CB2 receptor activation/deactivation.

In a further aspect, the present invention relates to a method for CB1 and/or CB2 receptor activation/deactivation, more specifically treatment of a medical condition that benefits from CB1 and/or CB2 receptor agonist/antagonist treatment, e.g., cancer, in a subject in need thereof, said method comprising administering to said subject a therapeutically effective amount of a compound as defined above, i.e., a THCA or CBDA derivative of the formula I, or an enantiomer, diastereomer, racemate, or pharmaceutically acceptable salt thereof.

### BRIEF DESCRIPTION OF THE DRAWINGS

FIG. 1 shows cell viability curve for PANC-1 and A549 at different cell numbers per well, after 2 hours with XTT in a 96-well plate.

### DETAILED DESCRIPTION OF THE PREFERRED EMBODIMENT

In one aspect, the present invention provides a compound, more particularly a THCA or CBDA derivative, of the formula I as defined above, or an enantiomer, diastereomer, racemate, or pharmaceutically acceptable salt thereof.

In certain embodiments, the compound disclosed herein is a THCA or CBDA derivative, wherein R₁ is H or R₅; and R₂ is —CN, —C(O)N(R₇)₂, —C(O)S—R₇, —CH₂N(R₇)₂, or a 5-6-membered aliphatic or aromatic heterocyclyl containing 1-4 heteroatoms and optionally substituted with one or more (C₁-C₄)alkyl groups, herein also identified as a THCA or CBDA derivative of the formula Ia. In other embodiments, the compound disclosed herein is a THCA or CBDA derivative, wherein R₁ is —C(O)— or absent; R₂ is —C(O)NR₇—, and R₁ and R₂ together with the atoms to which they are attached form a 5- or 6-membered heterocyclic ring, herein also identified as a THCA or CBDA derivative of the formula Ib (R₁ is —C(O)—) or Ic (R₁ is absent), respectively (Table 1).

TABLE 1
THCA and CBDA derivatives of the formulae Ia, Ib and Ic

| | |
|---|---|
| 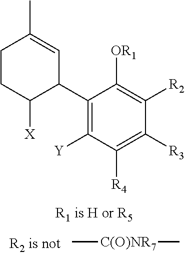 | Ia |
| 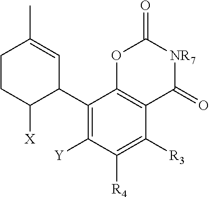 | Ib |
| 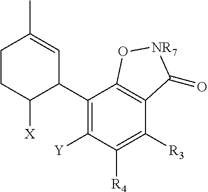 | Ic |

The term “halogen” as used herein refers to a halogen and includes fluoro, chloro, bromo, and iodo, but it is preferably fluoro or chloro.

The term “alkyl” as used herein typically means a linear or branched hydrocarbon radical having, e.g., 1-12 carbon atoms and includes methyl, ethyl, n-propyl, isopropyl, n-butyl, sec-butyl, isobutyl, tert-butyl, n-pentyl, isoamyl, 2,2-dimethylpropyl, n-hexyl, n-heptyl, n-octyl, n-nonyl, n-decyl, n-undecyl, n-dodecyl, and the like. Preferred are (C₁-C₈)alkyl groups, more preferably (C₁-C₆)alkyl groups, most preferably methyl, ethyl, n-propyl, isopropyl, n-butyl, sec-butyl, isobutyl, and tert-butyl. The alkyl may be substituted with one or more groups each independently selected from halogen, —OH, —NH₂, —O(C₁-C₁₂)alkyl, —(C₃-C₁₀)cycloalkyl, —(C₃-C₂)heterocyclyl, —(C₆-C₂)aryl, or —N(R₈)₂ wherein R is —(C₁-C₁₂)alkyl, —(C₁-C₁₂)haloalkyl, —(C₃-C₁₀)cycloalkyl, —(C₁-C₁₂)alkyl-aryl, —(C₁-C₁₂)alkyl-heterocyclyl, —(C₆-C₁₂)aryl, or —(C₃-C₁₂)heterocyclyl. The term “haloalkyl” thus typically means an alkyl as defined herein, substituted with one or more groups each independently selected from halogen, e.g., flouro.

The terms “alkenyl” and “alkynyl” as used herein typically mean straight and branched hydrocarbon radicals having, e.g., 2-12 carbon atoms and at least one double or triple bond, respectively, and include ethenyl, propenyl, 3-buten-1-yl, 2-ethenylbutyl, 3-octen-1-yl, 3-nonenyl, 3-decenyl, and the like, and ethynyl, propynyl, 2-butyn-1-yl, 3-pentyn-1-yl, 3-hexynyl, 3-octynyl, 4-decynyl, and the like. C₂-C₈ alkenyl and alkynyl radicals are preferred, more preferably C₂-C₆ alkenyl and alkynyl.

The term “cycloalkyl” as used herein means a mono- or bicyclic saturated hydrocarbyl group having, e.g., 3-10 carbon atoms such as cyclopropyl, cyclobutyl, cyclopentyl, cyclohexyl, cycloheptyl, adamantly, and the like, that may be substituted, e.g., by one or more alkyl groups.

The term “aryl” as used herein denotes an aromatic carbocyclic group having 6-14, preferably 6-12, carbon atoms consisting of a single ring or multiple rings either condensed or linked by a covalent bond such as, but not limited to, phenyl, naphthyl, phenanthryl, and biphenyl.

The term “heterocyclic ring” denotes a mono- or poly-cyclic aliphatic or aromatic ring of 3-12, preferably 5-6, atoms containing at least one carbon atom and one to four heteroatoms selected from sulfur, oxygen or nitrogen, which may be saturated or unsaturated, i.e., containing at least one unsaturated bond, and substituted, e.g., with one or more (C₁-C₄)alkyl groups such as methyl, ethyl, n-propyl, isopropyl, n-butyl, sec-butyl, isobutyl, and tert-butyl. Particular such heterocyclic rings contain one or more nitrogen atoms and are optionally substituted as defined hereinabove. The term “heterocyclyl” as used herein refers to any univalent radical derived from a heterocyclic ring as defined herein by removal of hydrogen from any ring atom. Examples of such radicals include, without limiting, pyridinyl, pyrimidinyl, aziridinyl, piperidinyl, pyrrolidinyl, azepinyl, morpholinyl such as 4-morpholinyl, oxazolyl, dihydrooxazolyl, oxadiazolyl; imidazolyl, imidazolinyl, dihydroimidazolyl, pyrazolyl, triazolyl, tetrazolyl, tetrazinyl, thiadiazolyl, piperazinyl, dihydroindolyl, quinolinyl, isoquinolinyl, tetrahydropirydinyl, oxapinyl, azacyclooctanyl, azaoxacyclooctanyl and azathiocyclooctanyl).

In certain embodiments, the present invention provides a compound of the formula I as defined above, wherein R₁ is absent, H, or —C(O)—. In certain particular such embodiments, R₁ is absent. In other particular such embodiments, R₁ is H. In further particular such embodiments, R₁ is —C(O)—.

In certain embodiments, the present invention provides a compound of the formula I as defined above, wherein R₂ is —CN, —C(O)NR₇—, —C(O)N(R₇)₂, —C(O)S—R₇, —CH₂N(R₇)₂, or a 5-6-membered aliphatic or aromatic heterocyclyl containing 1-4 heteroatoms and optionally substituted with one or more (C₁-C₄)alkyl groups. In some particular such embodiments, R₂ is —CN. In other particular such embodiments, R₂ is —C(O)NR₇—. In further particular such embodiments, R₂ is —C(O)N(R₇)₂. In still other particular such embodiments, R₂ is —C(O)S—R₇. In yet other particular such embodiments, R₂ is —CH₂N(R₇)₂. In still further particular such embodiments, R₂ is a 5-6-membered aliphatic or aromatic heterocyclyl containing 1-4 heteroatoms and optionally substituted with one or more (C₁-C₄)alkyl groups.

In certain embodiments, the present invention provides a compound of the formula I as defined above, wherein R₃ is —(C₁-C₈)alkyl. Particular such embodiments are those wherein R₃ is pentyl.

In certain embodiments, the present invention provides a compound of the formula I as defined above, wherein R₄ is H, or halogen such as fluoro or chloro.

In certain embodiments, the present invention provides a compound of the formula I as defined above, wherein R₅ is —(C₁-C₈)alkyl, preferably —(C₁-C₆)alkyl, or —(C₁-C₈)haloalkyl, preferably —(C₁-C₆)haloalkyl.

In certain embodiments, the present invention provides a compound of the formula I as defined above, wherein R₇ each independently is H, —OR₈, —N(R₅)₂, —N═C(R₈)₂, —NHC(O)R₈, —(C₁-C₈)alkyl substituted with one or more groups each independently selected from —OH, —N(R₉)₂, —O(C₁-C₈)alkyl, —(C₃-C₁₀)cycloalkyl, and —(C₃-C₁₂)heterocyclyl, or phenyl optionally substituted with one or more groups each independently selected from —OH, —N(R₉)₂, —O(C₁-C₈)alkyl, —(C₃-C₁₀)cycloalkyl, and —(C₃-C₁₂)heterocyclyl, or two R₇ together with the nitrogen atom to which they are attached form a 5- or 6-membered aliphatic or aromatic heterocyclic ring containing 1-4 heteroatoms; and R₈ and R₉ each independently is H, —(C₁-C₈)alkyl, preferably —(C₁-C₆)alkyl, —(C₁-C₈)haloalkyl, preferably —(C₁-C₆)haloalkyl, or —(C₃-C₁₀)cycloalkyl.

In certain embodiments, the present invention provides a compound of the formula I as defined above, wherein R₁ is absent, H, or —C(O)—; R₂ is —CN, —C(O)NR₇—, —C(O)N(R₇)₂, —C(O)S—R₇, —CH₂N(R₇)₂, or a 5-6-membered aliphatic or aromatic heterocyclyl containing 1-4 heteroatoms and optionally substituted with one or more (C₁-C₄)alkyl groups; R₃ is —(C₁-C₈)alkyl, preferably pentyl; R₄ is H, or halogen such as fluoro or chloro; R₇ each independently is H, —OR₅, —N(R₈)₂, —N═C(R₈)₂, —NHC(O)R₅, —(C₁-C₈)alkyl substituted with one or more groups each independently selected from —OH, —N(R₉)₂, —O(C₁-C₈)alkyl, —(C₃-C₁₀)cycloalkyl, and —(C₃-C₁₂)heterocyclyl, or phenyl optionally substituted with one or more groups each independently selected from —OH, —N(R₉)₂, —O(C₁-C₈)alkyl, —(C₃-C₁₀)cycloalkyl, and —(C₃-C₁₂)heterocyclyl, or two R₇ together with the nitrogen atom to which they are attached form a 5- or 6-membered aliphatic or aromatic heterocyclic ring containing 1-4 heteroatoms; and R₈ and R₉ each independently is H, —(C₁-C₈)alkyl, preferably —(C₁-C₆)alkyl, —(C₁-C₈)haloalkyl, preferably —(C₁-C₆)haloalkyl, or —(C₃-C₁₀)cycloalkyl. In some particular such embodiments, disclosed herein is a THCA or CBDA derivative of the formula Ia, wherein R₁ is H; and R₂ is —CN, —C(O)N(R₇)₂, —C(O)S—R₇, —CH₂N(R₇)₂, or a 5-6-membered aliphatic or aromatic heterocyclyl containing 1-4 heteroatoms and optionally substituted with one or more (C₁-C₄)alkyl groups. In other particular such embodiments, disclosed herein is a THCA or CBDA derivative of the formula Ib or Ic, wherein R₁ is —C(O)— or absent; R₂ is —C(O)NR₇—, and R₁ and R₂ together with the atoms to which they are attached form a 5- or 6-membered heterocyclic ring.

In certain embodiments, the compound disclosed herein is a THCA derivative, i.e., a compound of the formula I as defined in any one of the embodiments above, wherein X is the diradical

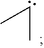

and Y is —O—, and together with X and the carbon atoms to which they are attached form a dihydropyran ring.

Specific such THCA derivatives of the formula Ia are those wherein: (i) R₁ is H; R₂ is —C(O)N(R₇)₂; R₃ is pentyl; R₄ is H; and R₇ each is H (herein identified compound Ia₁); (ii) R₁ is H; R₂ is —C(O)N(R₇)₂; R₃ is pentyl; R₄ is H; one of R₇ is H; and the other one of R₇ is 2-hydroxyethyl (herein identified compound Ia₃); (iii) R₁ is H; R₂ is —C(O)N(R₇)₂; R₃ is pentyl; R₄ is H; one of R₇ is H; and the other one of R₇ is 2-methoxyethyl (herein identified compound Ia₄); (iv) R₁ is H; R₂ is —C(O)N(R₇)₂; R₃ is pentyl; R₄ is H; one of R₇ is H; and the other one of R₇ is cyclopropylmethyl (herein identified compound Ia₅); (v) R₁ is H; R₂ is —C(O)N(R₇)₂; R₃ is pentyl; R₄ is H; one of R₇ is H; the other one of R₇ is 2-(dimethylamino)ethyl (herein identified compound Ia₆); (vi) R₁ is H; R₂ is —C(O)N(R₇)₂; R₃ is pentyl; R₄ is H; one of R₇ is H; the other one of R₇ is 2-aminoethyl (herein identified compound Ia₇); (vii) R₁ is H; R₂ is —C(O)N(R₇)₂; R₃ is pentyl; R₄ is H; one of R₇ is H; the other one of R₇ is 2-(dimethylamino)propyl (herein identified compound Ia₈); (viii) R₁ is H; R₂ is —C(O)N(R₇)₂; R₃ is pentyl; R₄ is H; one of R₇ is H; and the other one of R₇ is 3-(4-morpholinyl)propyl (herein identified compound Ia₉); (ix) R₁ is H; R₂ is —C(O)N(R₇)₂; R₃ is pentyl; R₄ is H; one of R₇ is H; and the other one of R₇ is 2-(4-morpholinyl)ethyl (herein identified compound Ia₁₀); (x) R₁ is H; R₂ is —C(O)N(R₇)₂; R₃ is pentyl; R₄ is H; one of R₇ is H; and the other one of R₇ is 4-pyridinylmethyl (herein identified compound Ia₁₁); (xi) R₁ is H; R₂ is —C(O)N(R₇)₂; R₃ is pentyl; R₄ is F; one of R₇ is H; and the other one of R₇ is cyclopropylmethyl (herein identified compound Ia₁₂); (xii) R₁ is H; R₂ is —C(O)N(R₇)₂; R₃ is pentyl; R₄ is H; one of R₇ is H; the other one of R₇ is —N(R₈)₂; and R₅ each is H (herein identified compound Ia₁₃); (xiii) R₁ is H; R₂ is —C(O)N(R₇)₂; R₃ is pentyl; R₄ is H; one of R₇ is H; the other one of R₇ is —NHC(O)R₈; and R₈ is methyl (herein identified compound Ia₁₄); (xiv) R₁ is H; R₂ is —C(O)N(R₇)₂; R₃ is pentyl; R₄ is H; one of R₇ is H; the other one of R₇ is —NHC(O)R₈; R₈ is —N(R₉)₂; one of R₉ is H; and the other one of R₉ is adamantyl (herein identified compound Ia₁₅); (xv) R₁ is H; R₂ is —C(O)N(R₇)₂; R₃ is pentyl; R₄ is H; one of R₇ is H; the other one of R₇ is —N═C(R₈)₂; one of R₈ is H; and the other one of R₅ is pyrimidin-2-yl (herein identified compound Ia₁₆); (xvi) R₁ is H; R₂ is —C(O)N(R₇)₂; R₃ is pentyl; R₄ is H; one of R₇ is H; and the other one of R₇ is 4-(4-morpholinyl)phenyl (herein identified compound Ia₁₇); (xvii) R₁ is H; R₂ is —C(O)N(R₇)₂; R₃ is pentyl; R₄ is H; one of R₇ is H; the other one of R is —OR₈; and R₈ is H (herein identified compound Ia₁₈); (xviii) R₁ is H; R₂ is —C(O)N(R₇)₂; R₃ is pentyl; R₄ is H; and the two R₇ together with the nitrogen atom to which they are attached form morpholinyl (herein identified compound Ia₁₉); (xix) R₁ is H; R₂ is —C(O)N(R₇)₂; R₃ is pentyl; R₄ is H; and the two R₇ together with the nitrogen atom to which they are attached form imidazolyl (herein identified compound Ia₂₀); (xx) R₁ is H; R₂ is —CH₂N(R₇)₂; R₃ is pentyl; R₄ is H; one of R₇ is H; and the other one of R₇ is 3-(4-morpholinyl)propyl (herein identified compound Ia₂₁); (xxi) R₁ is H; R₂ is —CN; R₃ is pentyl; and R₄ is H (herein identified compound Ia₂₂); (xxii) R₁ is H; R₂ is 2-imidazoline-2-yl; R₃ is pentyl; R₄ is H (herein identified compound Ia₂₃); (xxiii) R₁ is H; R₂ is 5-methyl-1,3,4-oxadiazol-2-yl; R₃ is pentyl; R₄ is H (herein identified compound Ia₂₄); (xxiv) R₁ is H; R₂ is —C(O)SR₇; R₃ is pentyl; R₄ is H; and R₇ is H (herein identified compound Ia₂₅); (xxv) R₁ is H; R₂ is —C(O)N(R₇)₂; R₃ is pentyl; R₄ is H; one of R₇ is H; and the other one of R₇ is —CH₂—CH₂—N(R₉)₂; one of R₉ is H; and the other one of R₉ is pyrimidin-2-yl (herein identified compound Ia₃₁); (xxvi) R₁ is H; R₂ is —C(O)N(R₇)₂; R₃ is pentyl; R₄ is H; one of R₇ is H; and the other one of R₇ is 2-(pyrrolidine-1-yl)ethyl (herein identified compound Ia₃₂); or (xxvii) R₁ is H; R₂ is —C(O)N(R₇)₂; R₃ is pentyl; R₄ is H; one of R₇ is H; and the other one of R₇ is 2-(1H-imidazol-1-yl)ethyl (herein identified compound Ia₃₃) (Table 2). In an additional THCA derivative exemplified herein, R₁ is H; R₂ is —C(O)N(R₇)₂; R₃ is pentyl; R₄ is H; one of R₇ is H; and the other one of R₇ is 2-methylpropyl (herein identified compound Ia₂) (Table 2).

In a specific such THCA derivative of the formula Ib, R₁ is —C(O)—; R₂ is —C(O)NR₇—; R₃ is pentyl; R₄ is H; and R₇ is 2-methoxyethyl (herein identified compound Ib₁) (Table 2).

In a specific such THCA derivative of the formula Ic, R₁ is absent; R₂ is —C(O)NR₇—; R₃ is pentyl; R₄ is H; and R₇ is H (herein identified compound Ic₁) (Table 2).

TABLE 2
Specific THCA derivatives of the formula I exemplified herein

| | |
|---|---|
| 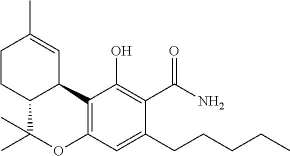 [SMILES: CCCCCc1cc2c(c(O)c1C(N)=O)[C@@H]1C=C(C)CC[C@H]1C(C)(C)O2] | Ia₁ |
| 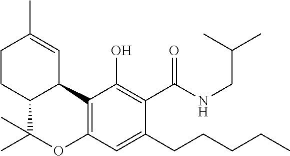 [SMILES: CCCCCc1cc2c(c(O)c1C(=O)NCC(C)C)[C@@H]1C=C(C)CC[C@H]1C(C)(C)O2] | Ia₂ |
| 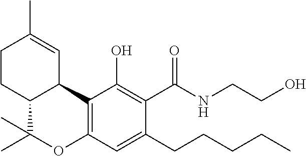 [SMILES: CCCCCc1cc2c(c(O)c1C(=O)NCCO)[C@@H]1C=C(C)CC[C@H]1C(C)(C)O2] | Ia₃ |
| 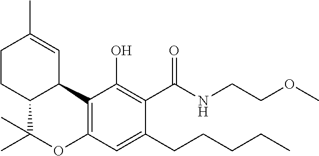 [SMILES: CCCCCc1cc2c(c(O)c1C(=O)NCCOC)[C@@H]1C=C(C)CC[C@H]1C(C)(C)O2] | Ia₄ |
| 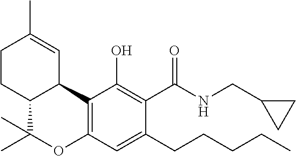 [SMILES: CCCCCc1cc2c(c(O)c1C(=O)NCC1CC1)[C@@H]1C=C(C)CC[C@H]1C(C)(C)O2] | Ia₅ |
| 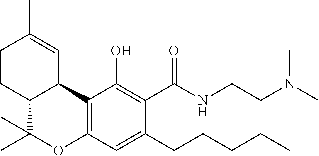 [SMILES: CCCCCc1cc2c(c(O)c1C(=O)NCCN(C)C)[C@@H]1C=C(C)CC[C@H]1C(C)(C)O2] | Ia₆ |
| 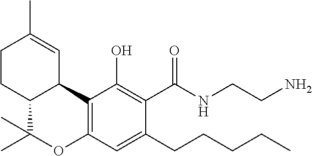 [SMILES: CCCCCc1cc2c(c(O)c1C(=O)NCCN)[C@@H]1C=C(C)CC[C@H]1C(C)(C)O2] | Ia₇ |
| 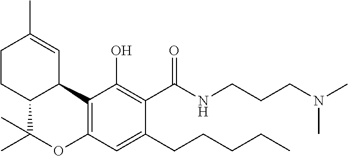 [SMILES: CCCCCc1cc2c(c(O)c1C(=O)NCCCN(C)C)[C@@H]1C=C(C)CC[C@H]1C(C)(C)O2] | Ia₈ |
| 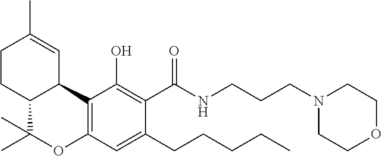 [SMILES: CCCCCc1cc2c(c(O)c1C(=O)NCCCN1CCOCC1)[C@@H]1C=C(C)CC[C@H]1C(C)(C)O2] | Ia₉ |
| 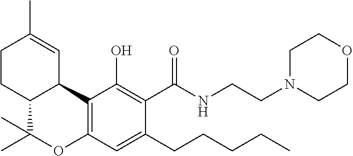 [SMILES: CCCCCc1cc2c(c(O)c1C(=O)NCCN1CCOCC1)[C@@H]1C=C(C)CC[C@H]1C(C)(C)O2] | Ia₁₀ |
| 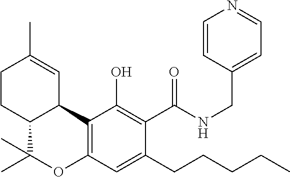 [SMILES: CCCCCc1cc2c(c(O)c1C(=O)NCc1ccncc1)[C@@H]1C=C(C)CC[C@H]1C(C)(C)O2] | Ia₁₁ |
| 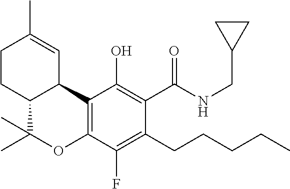 [SMILES: CCCCCc1c(F)c2c(c(O)c1C(=O)NCC1CC1)[C@@H]1C=C(C)CC[C@H]1C(C)(C)O2] | Ia₁₂ |
| 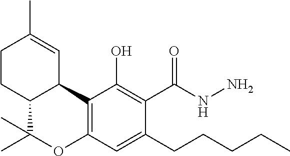 [SMILES: CCCCCc1cc2c(c(O)c1C(=O)NN)[C@@H]1C=C(C)CC[C@H]1C(C)(C)O2] | Ia₁₃ |
| 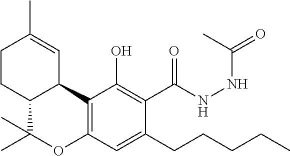 [SMILES: CCCCCc1cc2c(c(O)c1C(=O)NNC(C)=O)[C@@H]1C=C(C)CC[C@H]1C(C)(C)O2] | Ia₁₄ |
| 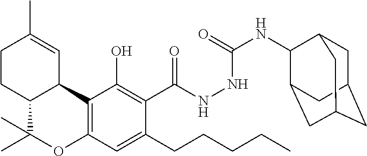 [SMILES: CCCCCc1cc2c(c(O)c1C(=O)NNC(=O)NC1C3CC4CC(C3)CC1C4)[C@@H]1C=C(C)CC[C@H]1C(C)(C)O2] | Ia₁₅ |
| 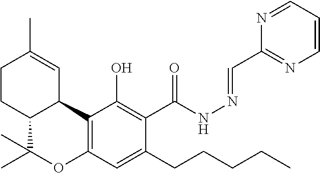 [SMILES: CCCCCc1cc2c(c(O)c1C(=O)N/N=C/c1ncccn1)[C@@H]1C=C(C)CC[C@H]1C(C)(C)O2] | Ia₁₆ |
| 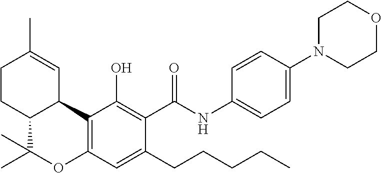 [SMILES: CCCCCc1cc2c(c(O)c1C(=O)Nc1ccc(N3CCOCC3)cc1)[C@@H]1C=C(C)CC[C@H]1C(C)(C)O2] | Ia₁₇ |
| 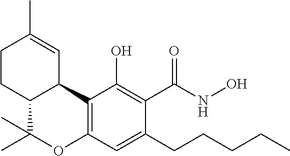 [SMILES: CCCCCc1cc2c(c(O)c1C(=O)NO)[C@@H]1C=C(C)CC[C@H]1C(C)(C)O2] | Ia₁₈ |
| 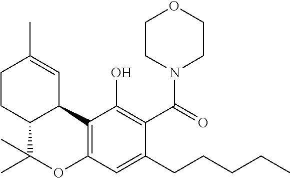 [SMILES: CCCCCc1cc2c(c(O)c1C(=O)N1CCOCC1)[C@@H]1C=C(C)CC[C@H]1C(C)(C)O2] | Ia₁₉ |
| 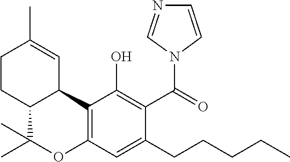 [SMILES: CCCCCc1cc2c(c(O)c1C(=O)n1ccnc1)[C@@H]1C=C(C)CC[C@H]1C(C)(C)O2] | Ia₂₀ |
| 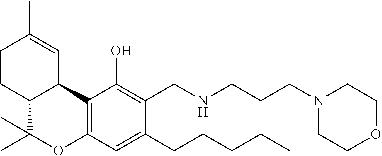 [SMILES: CCCCCc1cc2c(c(O)c1CNCCCN1CCOCC1)[C@@H]1C=C(C)CC[C@H]1C(C)(C)O2] | Ia₂₁ |
| 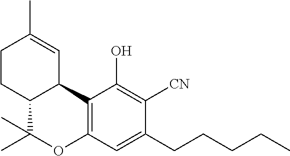 [SMILES: CCCCCc1cc2c(c(O)c1C#N)[C@@H]1C=C(C)CC[C@H]1C(C)(C)O2] | Ia₂₂ |
| 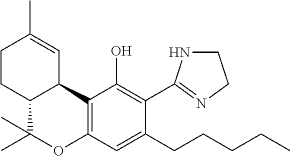 [SMILES: CCCCCc1cc2c(c(O)c1C1=NCCN1)[C@@H]1C=C(C)CC[C@H]1C(C)(C)O2] | Ia₂₃ |
| 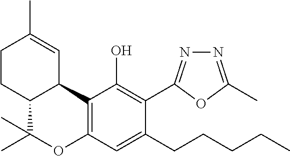 [SMILES: CCCCCc1cc2c(c(O)c1-c1nnc(C)o1)[C@@H]1C=C(C)CC[C@H]1C(C)(C)O2] | Ia₂₄ |
| 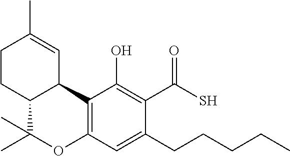 [SMILES: CCCCCc1cc2c(c(O)c1C(=O)S)[C@@H]1C=C(C)CC[C@H]1C(C)(C)O2] | Ia₂₅ |
| 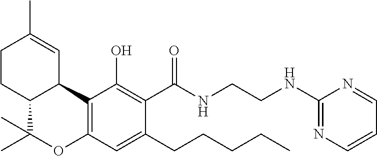 [SMILES: CCCCCc1cc2c(c(O)c1C(=O)NCCNc1ncccn1)[C@@H]1C=C(C)CC[C@H]1C(C)(C)O2] | Ia₃₁ |
| 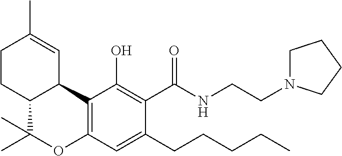 [SMILES: CCCCCc1cc2c(c(O)c1C(=O)NCCN1CCCC1)[C@@H]1C=C(C)CC[C@H]1C(C)(C)O2] | Ia₃₂ |
| 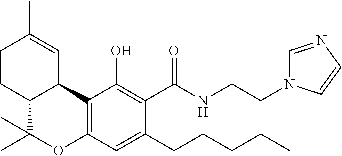 [SMILES: CCCCCc1cc2c(c(O)c1C(=O)NCCn1ccnc1)[C@@H]1C=C(C)CC[C@H]1C(C)(C)O2] | Ia₃₃ |
| 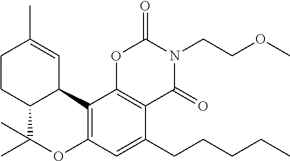 [SMILES: CCCCCc1cc2c(c3oc(=O)n(CCOC)c(=O)c13)[C@@H]1C=C(C)CC[C@H]1C(C)(C)O2] | Ib₁ |
| 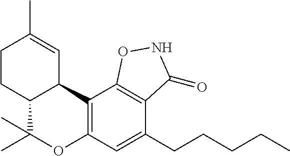 [SMILES: CCCCCc1cc2c(c3o[nH]c(=O)c13)[C@@H]1C=C(C)CC[C@H]1C(C)(C)O2] | Ic₁ |

In certain embodiments, the compound disclosed herein is a CBDA derivative, i.e., a compound of the formula I as defined in any one of the embodiments above, wherein X is the radical

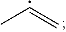

Y is —OH or —OR₅; and R₅ is —(C₁-C₈)alkyl, or —(C₁-C₈)haloalkyl. Particular such CBDA derivatives are those wherein Y is —OH.

Specific such CBDA derivatives of the formula Ia are those wherein: (i) R₁ is H; R₂ is —C(O)N(R₇)₂; R₃ is pentyl; R₄ is H; one of R₇ is H; and the other one of R₇ is 2-(dimethylamino)ethyl (herein identified compound Ia₂₆); (ii) R₁ is H; R₂ is —C(O)N(R₇)₂; R₃ is pentyl; R₄ is H; one of R₇ is H; and the other one of R₇ is 2-(pyrrolidine-1-yl)ethyl (herein identified compound Ia₂₇); (iii) R₁ is H; R₂ is —C(O)N(R₇)₂; R₃ is pentyl; R₄ is H; one of R₇ is H; and the other one of R₇ is 3-(morpholin-4-yl)propyl (herein identified compound Ia₂₈); or (iv) R₁ is H; R₂ is —C(O)N(R₇)₂; R₃ is pentyl; R₄ is H; one of R₇ is H; the other one of R₇ is —N(R₅)₂; and R₅ each is H (herein identified compound Ia₃₀) (Table 3). In an additional CBDA derivative exemplified herein, R₁ is H; R₂ is —C(O)N(R₇)₂; R₃ is pentyl; R₄ is H; one of R₇ is H; and the other one of R₇ is 2-methylpropyl (herein identified compound Ia₂₉) (Table 3).

TABLE 3
Specific CBDA derivatives of the formula I exemplified herein

| | |
|---|---|
| 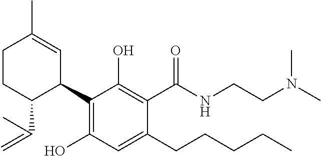 [SMILES: C=C(C)[C@@H]1CCC(C)=C[C@H]1c1c(O)cc(CCCCC)c(C(=O)NCCN(C)C)c1O] | Ia₂₆ |
| 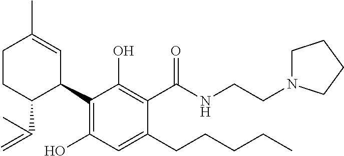 [SMILES: C=C(C)[C@@H]1CCC(C)=C[C@H]1c1c(O)cc(CCCCC)c(C(=O)NCCN2CCCC2)c1O] | Ia₂₇ |
| 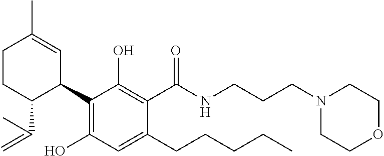 [SMILES: C=C(C)[C@@H]1CCC(C)=C[C@H]1c1c(O)cc(CCCCC)c(C(=O)NCCCN2CCOCC2)c1O] | Ia₂₈ |
| 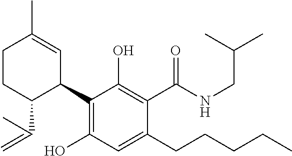 [SMILES: C=C(C)[C@@H]1CCC(C)=C[C@H]1c1c(O)cc(CCCCC)c(C(=O)NCC(C)C)c1O] | Ia₂₉ |
| 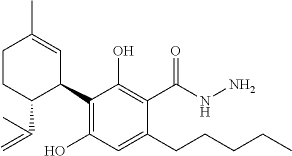 [SMILES: C=C(C)[C@@H]1CCC(C)=C[C@H]1c1c(O)cc(CCCCC)c(C(=O)NN)c1O] | Ia₃₀ |

The compounds of the formula I may have one or more asymmetric centers, and may accordingly exist both as enantiomers, i.e., optical isomers (R, S, or racemate, wherein a certain enantiomer may have an optical purity of 90%, 95%, 99% or more) and as diastereoisomers. The present invention encompasses all such enantiomers, isomers and mixtures thereof, as well as pharmaceutically acceptable salts thereof.

Optically active forms of the compounds of the formula I may be prepared using any method known in the art, e.g., by resolution of the racemic form by recrystallization techniques; by chiral synthesis; by extraction with chiral solvents; or by chromatographic separation using a chiral stationary phase. A non-limiting example of a method for obtaining optically active materials is transport across chiral membranes, i.e., a technique whereby a racemate is placed in contact with a thin membrane barrier, the concentration or pressure differential causes preferential transport across the membrane barrier, and separation occurs as a result of the non-racemic chiral nature of the membrane that allows only one enantiomer of the racemate to pass through. Chiral chromatography, including simulated moving bed chromatography, can also be used. A wide variety of chiral stationary phases are commercially available.

The THCA and CBDA derivatives of the present invention are expected to be effective in CB1 and/or CB2 receptor activation/deactivation, more particularly in all those clinical conditions wherein administration of THCA and CBDA as CB1 and/or CB2 receptor agonists/antagonists is of benefit. Examples of such conditions include, without being limited to, inflammatory diseases, conditions associated with pain as disclosed e.g., in WO2016044370, cancer and tumors (disclosed, e.g., in Soderstrom et al (2017) and WO2014198993; WO2016087649), brain and spinal cord diseases (disclosed, e.g., in WO2017178810, WO2016109624 and WO2015198209), skin diseases (disclosed, e.g., in US20180042890), immunological diseases including autoimmune diseases, neurologic diseases, neurodegenerative diseases or disorders, and neuroinflammatory conditions (disclosed, e.g., in CA2910206). The neuroprotective activities of the compounds of the invention is expected to be useful in preventing, alleviating or treating neurological disorders, neurodegenerative diseases or disorders, and neuroinflammatory conditions such as, but not limited to, stroke, migraine, cluster headache, epilepsy, Parkinson's disease, Alzheimer's disease, amyotrophic lateral sclerosis (ALS), Huntington's chorea, prion-associated diseases, poisoning of the central nervous system, motor disorders, muscle spasm and tremor, meningitis, encephalitis, cerebral ischemia and Guillain-Barre syndrome. The Examples section hereinafter shows that such compounds are capable of inhibiting cancer cell growth.

In another aspect, the present invention thus provides a pharmaceutical composition comprising a THCA or CBDA derivative of the formula I as defined in any one of the embodiments above, or an enantiomer, diastereomer, racemate, or pharmaceutically acceptable salt thereof, herein also referred to as “the active agent”, and a pharmaceutically acceptable carrier.

In certain embodiments, the pharmaceutical composition disclosed herein comprises a THCA derivative, i.e., a compound of the formula I as defined in any one of the embodiments above, wherein X is the diradical

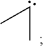

and Y is —O—, and together with X and the carbon atoms to which they are attached form a dihydropyran ring. Particular such THCA derivatives are those identified herein as compounds Ia₁, Ia₃₋₂₅, Ia₃₁₋₃₃, Ib₁, and Ic₁, shown in Table 2, wherein each one of the specific compounds represents a separate embodiment.

In other embodiments, the pharmaceutical composition disclosed herein comprises a CBDA derivative, i.e., a compound of the formula I as defined in any one of the embodiments above, wherein X is the radical

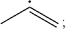

Y is —OH or —OR₅, but preferably —OH; and R₅ is —(C₁-C₈)alkyl, or —(C₁-C₈)haloalkyl. Particular such CBDA derivatives are those are those identified herein as compounds Ia₂₆₋₂₈, and Ia₃₀, shown in Table 3, wherein each one of the specific compounds represents a separate embodiment.

Pharmaceutical compositions according to the present invention may be prepared by conventional techniques, e.g., as described in Remington: The Science and Practice of Pharmacy, 19^{th} Ed., 1995. The compositions can be prepared, e.g., by uniformly and intimately bringing the active agent, i.e., the THCA or CBDA derivative disclosed herein, into association with a liquid carrier, a finely divided solid carrier, or both, and then, if necessary, shaping the product into the desired formulation. The compositions may be in the form of a liquid (e.g., solution, emulsion, or suspension), gel, cream, solid, semisolid, film, foam, lyophilizate, or aerosol, and may further include pharmaceutically and physiologically acceptable fillers, carriers, diluents or adjuvants, and other inert ingredients and excipients. In one embodiment, the pharmaceutical composition of the invention is formulated as nanoparticles or microparticles.

A pharmaceutical composition according to the present invention can be formulated for any suitable route of administration, e.g., for parenteral administration such as intravenous, intraarterial, intrathecal, intrapleural, intratracheal, intraperitoneal, intramuscular, subcutaneous, transdermal, or intradermal administration, topical administration, oral, sublingual, buccal, enteral, or rectal administration, or for inhalation. In particular embodiments, such a composition is formulated for intravenous or intraperitoneal administration, or for subcutaneous administration.

The pharmaceutical compositions of the invention, when formulated for oral administration, may be in any suitable form, e.g., tablets, troches, lozenges, aqueous, or oily suspensions, dispersible powders or granules, emulsions, hard or soft capsules, or syrups or elixirs. In certain embodiments, said tablets are in the form of matrix tablets in which the release of a soluble active agent(s) is controlled by having the active agent(s) diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other embodiments, the tablets are formulated as bi- or multi-layer tablets, made up of two or more distinct layers of granulation compressed together with the individual layers lying one on top of another, with each separate layer containing a different active agent. Bilayer tablets have the appearance of a sandwich since the edge of each layer or zone is exposed. In further embodiments, the compositions comprise the active agent(s) formulated for controlled release in microencapsulated dosage form, in which small droplets of the active agent(s) are surrounded by a coating or a membrane to form particles in the range of a few micrometers to a few millimeters.

Pharmaceutical compositions for oral administration might be formulated so as to inhibit the release of one or both of the active agents in the stomach, i.e., delay the release of one or both of the active agents until at least a portion of the dosage form has traversed the stomach, in order to avoid the acidity of the gastric contents from hydrolyzing the active agent. Particular such compositions are those wherein the active agent is coated by a pH-dependent enteric-coating polymer. Examples of pH-dependent enteric-coating polymer include, without being limited to, Eudragit® S (poly(methacrylicacid, methylmethacrylate), 1:2), Eudragit® L 55 (poly (methacrylicacid, ethylacrylate), 1:1), Kollicoat® (poly(methacrylicacid, ethylacrylate), 1:1), hydroxypropyl methylcellulose phthalate (HPMCP), alginates, carboxymethylcellulose, and combinations thereof. The pH-dependent enteric-coating polymer may be present in the composition in an amount from about 10% to about 95% by weight of the entire composition.

In certain embodiments, the invention provides a pharmaceutical composition for oral administration, which is solid and may be in the form of granulate, granules, grains, beads or pellets, mixed and filled into capsules or sachets, or compressed to tablets by conventional methods. In some particular embodiments, the pharmaceutical composition is in the form of a bi- or multilayer tablet, in which each one of the layers comprise one of the two active agents, and the layers are optionally separated by an intermediate, inactive layer, e.g., a layer comprising one or more disintegrants.

Another contemplated formulation is depot systems, based on biodegradable polymers. As the polymer degrades, the active agent(s) is slowly released. The most common class of biodegradable polymers is the hydrolytically labile polyesters prepared from lactic acid, glycolic acid, or combinations of these two molecules. Polymers prepared from these individual monomers include poly (D,L-lactide) (PLA), poly (glycolide) (PGA), and the copolymer poly (D,L-lactide-co-glycolide) (PLG).

Pharmaceutical compositions for oral administration may be prepared according to any method known to the art and may further comprise one or more agents selected from sweetening agents, flavoring agents, coloring agents and preserving agents in order to provide pharmaceutically elegant and palatable preparations. Tablets contain the active agents in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the invention may also be in the form of oil-in-water emulsion.

Useful dosage forms of the pharmaceutical compositions include orally disintegrating systems including, but not limited to, solid, semi-solid and liquid systems including disintegrating or dissolving tablets, soft or hard capsules, gels, fast dispersing dosage forms, controlled dispersing dosage forms, caplets, films, wafers, ovules, granules, buccal/mucoadhesive patches, powders, freeze dried (lyophilized) wafers, chewable tablets which disintegrate with saliva in the buccal/mouth cavity and combinations thereof. Useful films include, but are not limited to, single layer stand-alone films and dry multiple layer stand-alone films.

The pharmaceutical composition of the invention may comprise one or more pharmaceutically acceptable excipients. For example, a tablet may comprise at least one filler, e.g., lactose, ethylcellulose, microcrystalline cellulose, silicified microcrystalline cellulose; at least one disintegrant, e.g., cross-linked polyvinylpyrrolidinone; at least one binder, e.g., polyvinylpyridone, hydroxypropylmethyl cellulose; at least one surfactant, e.g., sodium laurylsulfate; at least one glidant, e.g., colloidal silicon dioxide; and at least one lubricant, e.g., magnesium stearate.

Pharmaceutical compositions for rectal administration may be in any suitable form, e.g., a liquid or gel for injection into the lower bowel by way of the rectum using an enema, or formulated as a suppository, i.e., a solid dosage form for insertion into the rectum.

The pharmaceutical composition of the invention may be in the form of a sterile injectable aqueous or oleagenous suspension, which may be formulated according to the known art using suitable dispersing, wetting or suspending agents. The sterile injectable preparation may also be an injectable solution or suspension in a non-toxic parenterally acceptable diluent or solvent. Acceptable vehicles and solvents that may be employed include, without limiting, water, Ringer's solution, polyethylene glycol (PEG), 2-hydroxypropyl-β-cyclodextrin (HPCD), a surfactant such as Tween-80, and isotonic sodium chloride solution.

Pharmaceutical compositions according to the invention, when formulated for inhalation, may be in any suitable form, e.g., liquid or fine powder, and may be administered utilizing any suitable device known in the art, such as pressurized metered dose inhalers, liquid nebulizers, dry powder inhalers, sprayers, thermal vaporizers, electrohydrodynamic aerosolizers, and the like.

The pharmaceutical compositions of the invention may be administered, e.g., continuously, daily, twice daily, thrice daily or four times daily, for various duration periods, e.g., weeks, months, years, or decades. The dosages will depend on the state of the patient, and will be determined, from time to time, as deemed appropriate by the practitioner. For example, a physician or veterinarian could start doses of the active agents employed in the pharmaceutical composition at levels lower than required in order to achieve the desired therapeutic effect, and gradually increase the dosage until the desired effect is achieved.

In yet another aspect, the present invention relates to a THCA or CBDA derivative of the formula I as defined in any one of the embodiments above, or an enantiomer, diastereomer, racemate, or pharmaceutically acceptable salt thereof, for use in CB1 and/or CB2 receptor activation/deactivation, more specifically treatment of a medical condition that benefits from CB1 and/or CB2 receptor agonist/antagonist treatment.

In still another aspect, the present invention relates to use of a THCA or CBDA derivative of the formula I as defined in any one of the embodiments above, or an enantiomer, diastereomer, racemate, or pharmaceutically acceptable salt thereof, in the manufacture of a pharmaceutical composition for CB1 and/or CB2 receptor activation/deactivation, more specifically treatment of a medical condition that benefits from CB1 and/or CB2 receptor agonist/antagonist treatment.

In a further aspect, the present invention relates to a method for CB1 and/or CB2 receptor activation/deactivation, more specifically treatment of a medical condition that benefits from CB1 and/or CB2 receptor agonist/antagonist treatment, e.g., cancer, in a subject in need thereof, said method comprising administering to said subject a therapeutically effective amount of a THCA or CBDA derivative of the formula I as defined in any one of the embodiments above, or an enantiomer, diastereomer, racemate, or pharmaceutically acceptable salt thereof.

In specific embodiments, the THCA derivative administered according to the method disclosed herein is a compound selected from those herein identified as compounds Ia₁, Ia₃₋₂₅, Ia₃₁₋₃₃, Ib₁, and Ic₁, shown in Table 2; or those herein identified as compounds Ia₂₆₋₂₈, and Ia₃₀, shown in Table 3, wherein each one of the specific compounds represents a separate embodiment.

In certain embodiments, the subject administered with the compound of the present invention suffers from an inflammatory disease, pain or a condition associated therewith, brain or spinal cord disease, skin disease, immunological disease including autoimmune disease, neurologic disease, neurodegenerative disease or disorder, neuroinflammatory condition, or cancer. Non-limiting examples of cancer that can be treated according to the method disclosed herein include adenocarcinoma such as colon adenocarcinoma, prostate adenocarcinoma, and liver adenocarcinoma; carcinoma such as esophagus carcinoma, pancreas ductal carcinoma, breast ductal carcinoma, and lung carcinoma; multiple myeloma; brain glioma, or brain glioblastoma.

The term “subject” as used herein refers to any mammal, e.g., a human, non-human primate, horse, ferret, dog, cat, cow, and goat. In a preferred embodiment, the term “subject” denotes a human, i.e., an individual.

The term “treatment”, as used herein with respect to a medical condition (i.e., disease, disorder, or condition) that benefits from CB1 and/or CB2 receptor agonist treatment, refers to the administration of a therapeutically effective amount of an active agent, i.e., a THCA or CBDA derivative of the formula I, as defined herein, which is effective to ameliorate undesired symptoms associated with said medical condition; prevent the manifestation of such symptoms before they occur; slow down the progression of said medical condition; slow down the deterioration of symptoms; enhance the onset of remission period; slow down the irreversible damage caused in the progressive chronic stage of said medical condition; delay the onset of said progressive stage; lessen the severity or cure said medical condition; improve survival rate or more rapid recovery; and/or prevent said medical condition form occurring.

The term “therapeutically effective amount” as used herein with respect to the active agent administered according to the method of the invention refers to an amount of said active agent that upon administration under a particular regimen during a particular period of time, e.g., days, weeks, months or years, is sufficient to prevent, inhibit, ameliorate, or treat a medical condition (i.e., disease, disorder, or condition) that benefits from CB1 and/or CB2 receptor agonist treatment, occurring in the body of the subject administered with. The actual dosages of both the active agent administered may be varied so as to obtain amounts of said active agent that are effective to achieve the desired prophylactic/therapeutic response for a particular subject and mode of administration, without being toxic to the subject. The dosage level selected will depend upon a variety of factors including the activity of the active agent employed, the route of administration, the duration of the treatment, and other drugs, if any, used in addition to the active agent employed, as well as the age, sex and weight of the subject treated, and the severity/progression of the medical condition. In general, it may be presumed that for preventive treatment, lower doses will be needed, while higher doses will be required for treatment of subjects already showing pathological phenotypes of said medical condition.

Unless otherwise indicated, all numbers expressing quantities of ingredients and so forth used in the present description and claims are to be understood as being modified in all instances by the term “about”. Accordingly, unless indicated to the contrary, the numerical parameters set forth in this specification and attached claims are approximations that may vary by up to plus or minus 10% depending upon the desired properties sought to be obtained by the present invention.

The entire contents of each and all patents and patent publications listed above being herewith incorporated by reference in their entirety as if fully disclosed herein.

The invention will now be illustrated by the following non-limiting Examples.

### EXAMPLES

### Materials and Methods

In the examples, reactions were provided in appropriate solvents as THF, ACN and DMF, as well as reagents obtained from Sigma Aldrich Corp. The completion of the reaction was monitored by TLC, the mixture was washed twice with a solution of citric acid and then once with brine. The organic phase was separated, dried and the crude product was isolated and purified by column chromatography on silica gel with EA in hexane or PE as the eluent. The level of purity was examined by HPLC and LCMS. All compounds were characterized by mass and NMR spectroscopy by 400 MHz (VARIAN). These methods were consisted with assigned structures. In the following examples CBDA and THCA were used for the preparation of novel compounds of the invention. These acids were obtained from plant material including technical hemp and Cannabis sativa using extraction and chromatographic methods for their isolation and purification.

Compounds of the formula I wherein R₂ is —C(O)N(R₇)₂ were generally synthesized using CDI as a coupling reagent, as depicted in Scheme 1. The use of CDI enables carrying out the reaction without isolating the intermediate product.

Compounds of the formula I wherein R₂ is a heterocyclyl were generally synthesized by the cyclisation reactions of the corresponding amides and hydrazides, as depicted in Scheme 2.

Monosubstituted amides of the formula I can be used as precursors in the synthesis of other derivatives by reaction with triphosgene, as depicted in Scheme 3.

Reaction of THCA with hydroxylamine in the presence of excess of CDI resulted in not only to hydroxamic acid but to corresponding isoxazole (compound Ic₁), as depicted in Scheme 4. Such cyclisation of o-OH hydroxamic acids was described in WO2005089753.

Reduction of amide (compound Ia₉) with LiAlH₄ in THF provides the corresponding amine (compound Ia₂₀) according to reduction of o-hydroxybenzamides (Assimomytis et al., Synlett, 2009, 17, 2777-2782), as depicted in Scheme 5.

Reaction of THCA with Lawesson's reagent in ACN resulted in the corresponding thioacid compound Ia₂₅ in a moderate yield, as depicted in Scheme 6.

Reaction of amide (compound Ia₅) with 1-fluoropyridinium tetrafluoroborate in DCM after resulted in flour derivative Ia₁₂, as depicted in Scheme 7.

Reaction of THCA with trimethylsilyl hydroxylamine (Bottaro et al., 1985) in the presence of CDI resulted after acidic hydrolysis in the corresponding hydroxamic acid Ia₁₈ in a moderate yield, as depicted in Scheme 8.

### Example 1. Synthesis of (6aR,0aR)-1-hydroxy-6,6,9-trimethyl-3-pentyl-6a,7,8,10a-tetrahydro-6H-benzo[c]chromene-2-carboxamide (Ia₁)

To a solution of THCA (191 mg, 0.53 mmol) in dry THF (5 ml) CDI (211 mg, 1.3 mmol) was added and the mixture was stirred for one hour. Then solution of ammonia in 1 ml THF (1 ml, 2.6 mmol) was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 31% (59 mg, 0.16 mmol). ¹H NMR (CD₃OH), δ: 0.88-0.92 (3H, t, CH); 1.06 (3H, s, CH); 1.29-1.36 (6H, m, CH); 1.42 (3H, s, CH); 1.54-1.59 (3H, m, CH); 1.64-1.70 (1H, m, CH); 1.66 (3H, s, CH); 1.95-1.98 (1H, m, CH); 2.15-2.17 (2H, m, CH); 2.64-2.79 (2H, t, CH); 3.18-3.21 (1H, d, CH); 6.20 (1H, s, CH); 6.19 (1 h, s, CH); 6.36 (1H, bs, NH). ¹³C NMR (CD₃OD), δ: 14.36, 19.45, 23.50, 25.19, 26.18, 27.84, 31.96, 32.21, 32.90, 34.98, 35.10, 47.36, 78.71, 111.49, 112.68, 112.67, 125.34, 134.18, 141.45, 157.45, 158.31, 175.31. Molecular ion observed [M-H]+=468 consistent with the molecular formula C₂₂H₃₁NO₃.

### Example 2. Synthesis of (6aR,0aR)-1-hydroxy-N-isobutyl-6,6,9-trimethyl-3-pentyl-6a,7,8,10a-tetrahydro-6H-benzo[c]chromene-2-carboxamide (Ia₂)

To a solution of THCA (138 mg, 0.38 mmol) in dry THF (5 ml) CDI (151 mg, 0.93 mmol) was added and the mixture was stirred for one hour. Then methylisopropylamine in 1 ml THF (148 mg, 2.02 mmol) was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 31% (49 mg, 0.11 mmol). ¹H NMR (CDCl₃), δ: 0.88-0.92 (3H, t, CH); 0.98-1.00 (6H, d, CH); 1.09 (3H, s, CH); 1.32-1.36 (6H, m, CH); 1.42 (3H, s, CH); 1.60-1.64 (7H, m, CH); 1.71-1.77 (2H, m, CH); 1.95-1.98 (1H, m, CH); 2.14-2.16 (2H, m, CH); 2.67-2.79 (2H, t, CH); 2.97-2.99 (2H, t, CH); 3.18-3.23 (1H, d, CH); 3.28-3.32 (2H, mm CH); 6.21 (1H, s, CH); 6.40 (1 h, s, CH); 6.36 (1H, bs, NH). ¹³C NMR (CDCl₃), δ: 14.09, 19.52, 20.24, 20.49, 22.61, 23.46, 25.16, 27.61, 30.47, 31.38, 32.01, 33.81, 35.05, 35.87, 47.55, 48.29, 78.29, 109.58, 110.59, 110.93, 123.97, 133.81, 139.34, 156.84, 160.25, 171.32. Molecular ion observed [M-H]⁺=414 consistent with the molecular formula C₂₆H₃₉NO₃.

### Example 3. Synthesis of (6aR,10aR)-1-hydroxy-N-(2-hydroxyethyl)-6,6,9-trimethyl-3-pentyl-6a,7,8,10a-tetrahydro-6H-benzo[c]chromene-2-carboxamide (Ia₃)

To a solution of THCA (261 mg, 0.72 mmol) in dry THF (4 ml) CDI (160 mg, 0.98 mmol) was added and the mixture was stirred for one hour. Then ethanolamine (95 mg, 1.33 mmol) was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 38% (112 mg, 0.27 mmol). ¹H NMR (CDCl₃, δ: 0.89 (3H, m, CH); 1.09 (3H, s, CH); 1.32-1.42 (8H, m, CH); 1.58-1.70 (6H, m, CH); 1.88-1.91 (2H, m, CH); 2.15-2.16 (2H, m, CH); 2.69-2.71 (2H, t, CH); 3.20-3.23 (1H, d, CH); 3.60-3.64 (2H, q, CH); 3.82-3.84 (2H, t, CH); 6.21 (1H, s, CH); 6.39 (1H, s, CH); 6.39 (1H, s, NH). ¹³C NMR (CDCl₃), δ: 14.11, 19.52, 22.58, 23.46, 25.15, 27.59, 31.26, 31.36, 31.97, 33.78, 35.06, 42.41, 45.84, 62.02, 78.31, 109.17, 110.58, 111.20, 123.92, 133.85, 139.79, 157.05, 160.28, 171.85. Molecular ion observed [M-H]⁺=402 consistent with the molecular formula C₂₄H₃₅NO.

### Example 4. Synthesis of (6aR,0aR)-1-hydroxy-N-(2-methoxyethyl)-6,6,9-trimethyl-3-pentyl-6a,7,8,10a-tetrahydro-6H-benzo[c]chromene-2-carboxamide (Ia₄)

To a solution of THCA (360 mg, 1 mmol) in dry THF (5 ml) CDI (210 mg, 1.29 mmol) was added and the mixture was stirred for one hour. Then 2-methohyethylamine (162 mg, 2.14 mmol) in THF (1 ml) was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 47% (205 mg, 0.48 mmol)). ¹H NMR (CD₃OD), δ: 0.83 (3H, t, CH); 0.98 (3H, s, CH); 1.22-1.37 (8H, m, CH); 1.43-1.55 (3H, m, CH); 1.55 (3H, s, CH); 1.86-1.89 (1H, m, CH); 2.08-2.10 (2H, m, CH); 2.51-2.59 (2H, m, CH); 3.11-3.14 (1H, d, CH); 3.31 (3H, s, CH); 3.44-3.49 (4H, m, CH); 6.11 (1H, s, CH); 6.27 (1H, s, CH). ¹³C NMR (CD₃OD), δ: 14.39, 17.98, 19.39, 22.09, 22.15, 23.50, 26.18, 27.84, 32.20, 32.28, 34.69, 35.02, 37.40, 40.45, 47.39, 58.93, 71.84, 78.55, 111.57, 116.0, 125.36, 134.21, 141.14, 156.99, 172.28. Molecular ion observed [M-H]⁺=416 consistent with the molecular formula C₂₅H₃₇NO₄.

### Example 5. Synthesis of (6aR,0aR)—N-(cyclopropylmethyl)-1-hydroxy-6,6,9-trimethyl-3-pentyl-6a,7,8,10a-tetrahydro-6H-benzo[c]chromene-2-carboxamide (Ia₅)

To a solution of THCA (161 mg, 0.44 mmol) in dry THF (5 ml) CDI (160 mg, 0.98 mmol) was added and the mixture was stirred for one hour. Then aminomethylcyclopropane (80 mg, 1.11 mmol) was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 45% (83 mg, 0.21 mmol). ¹H NMR (CDCl₃, δ: 0.25-0.29 (2H, q, CH); 0.55-0.60 (2H, m, CH); 0.90 (3H, t, CH); 1.02-1.08 (1H, m, CH); 1.09 (3H, s, CH); 1.34-1.38 (4H, m, CH); 1.40-1.43 (1H, m, CH); 1.43 (3H, s, CH); 1.64-1.70 (3H, m, CH); 1.65 (3H, s, CH); 1.88-1.90 (1H, m, CH); 2.15-2.16 (2H, m, CH); 2.69-2.73 (2H, m, CH); 3.20-3.28 (1H, m, CH); 3.29-3.37 (2H, m, CH); 6.05 (1H, s, NH); 6.21 (1H, s, CH); 6.40 (1H, s, CH); 11.73 (1H, s, OH). ¹³C NMR (CDCl₃), δ: 3.70, 10.62, 14.11, 22.65, 23.46, 25.16, 27.60, 30.46, 31.35, 31.36, 31.37, 32.10, 33.79, 35.16, 45.07, 45.86, 78.29, 109.37, 110.58, 111.08, 123.78, 139.53, 156.87, 160.34, 171.01. Molecular ion observed [M-H]⁺=412 consistent with the molecular formula C₂₆H₃₇NO₃.

### Example 6. Synthesis of (6aR,0aR)—N-(2-(dimethylamino)ethyl)-1-hydroxy-6,6,9-trimethyl-3-pentyl-6a,7,8,10a-tetrahydro-6H-benzo[c]chromene-2-carboxamide (Ia₆)

To a solution of THCA (339 mg, 0.94 mmol) in dry THF (5 ml) CDI (206 mg, 1.27 mmol) was added and the mixture was stirred for one hour. Then N,N-dimethylethylenediamine (120 mg, 1.36 mmol) was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 53% (205 mg, 0.47 mmol). ¹H NMR (CDCl₃, δ: 0.87-0.89 (3H, t, CH); 1.08 (3H, s, CH); 1.32-1.33 (4H, m, CH); 1.41 (3H, s, CH); 1.59-1.70 (3H, m, CH); 1.64 (3H, s, CH); 1.88-1.92 (1H, m, CH); 2.14-2.16 (2H, m, CH); 2.26 (6H, s, CH); 2.49-2.52 (2H, t, CH); 2.60-2.73 (2H, m, CH); 3.20-3.23 (1H, m, CH); 3.43-3.55 (2H, m, CH); 6.21 (1H, s, CH); 6.41 (1H, s, CH); 6.58 (1H, s, NH). ¹³C NMR (CDCl₃) δ: 14.16, 19.50, 22.72, 23.50, 25.19, 27.63, 31.28, 31.40, 32.12, 33.89, 34.87, 37.28, 45.03, 45.93, 57.34, 78.12, 110.39, 110.41, 110.73, 124.18, 133.66, 140.13, 156.62, 159.64, 170.90. Molecular ion observed [M-H]⁺=429 consistent with the molecular formula C₂₆H₄₀N₂O₃.

### Example 7. Synthesis of (6aR,10aR)—N-(2-aminoethyl)-1-hydroxy-6,6,9-trimethyl-3-pentyl-6a,7,8,10a-tetrahydro-6H-benzo[c]chromene-2-carboxamide (Ia₇)

To a solution of THCA (185 mg, 0.51 mmol) in dry THF (6 ml) CDI (229 mg, 1.41 mmol) was added and the mixture was stirred for one hour. Then ethylenediamine (145 mg, 2.1 mmol) in 1 ml THF was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 80% (165 mg, 0.41 mmol). ¹H NMR (CD₃OD), δ: 0.88-0.92 (3H, t, CH); 1.05 (3H, s, CH); 1.32-1.37 (8H, m, CH); 1.42 (3H, s, CH); 1.54-1.62 (4H, m, CH), 1.65 (3H, s, CH); 1.94-1.97 (1H, m, CH); 2.13-2.19 (1H, m, CH); 2.56-2.60 (2H, t, CH); 2.84-2.87 (2H, t, CH); 3.19-3.22 (2H, bd, CH); 3.44-3.47 (2H, t, CH); 6.16 (1H, s, CH); 6.35 (1 h, s, CH); ¹³C NMR (CD₃OD), δ: 14.36, 19.32, 23.47, 26.20, 27.84, 32.03, 32.21, 32.92, 34.52, 35.08, 41.82, 42.71, 47.50, 64.85, 67.78, 78.38, 110.98, 111.79, 117.59, 125.46, 134.89, 140.86, 156.80, 173.93. Molecular ion observed [M-H]⁺=401 consistent with the molecular formula C₂₄H₃₆N₂O₃.

### Example 8. (6aR,0aR)—N-(3-(dimethylamino)propyl)-1-hydroxy-6,6,9-trimethyl-3-pentyl-6a,7,8,10a-tetrahydro-6H-benzo[c]chromene-2-carboxamide (Ia₈)

To a solution of THCA (119 mg, 0.33 mmol) in dry THF (5 ml) CDI (102 mg, 0.94 mmol) was added and the mixture was stirred for one hour. Then N¹,N¹-dimethylpropane-1,3-diamine (150 mg, 1.47 mmol) in 5 ml THF was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 43% (64 mg, 0.14 mmol). ¹H NMR (CDCl₃), δ: 0.87-0.91 (3H, t, CH); 1.09 (3H, s, CH); 1.26-1.33 (5H, m, CH); 1.40 (3H, s, CH); 1.57-1.61 (1H, m, CH), 1.57-1.61 (3H, m, CH); 1.66-1.69 (4H, m, CH); 1.79-1.83 (2H, m, CH); 1.90-1.94 (1H, m, CH); 2.16-2.17 (2H, m, CH); 2.50-2.54 (2H, m, CH); 2.68-2.75 (2H, m, CH); 3.21-3.24 (1H, d, CH); 3.54-3.60 (2H, m, CH); 6.24 (1H, s, CH); 6.21 (1H, s, CH); 6.41 (1H, s, CH); 7.33 (1H, d, NH). Molecular ion observed [M-H]⁺=519 consistent with the molecular formula C₂₇H₄₂N₂O₃.

### Example 9. Synthesis of (6aR,0aR)-1-hydroxy-6,6,9-trimethyl-N-(3-morpholinopropyl)-3-pentyl-6a,7,8,10a-tetrahydro-6H-benzo[c]chromene-2-carboxamide (Ia₉)

To a solution of THCA (177 mg, 0.49 mmol) in dry THF (5 ml) CDI (165 mg, 1.03 mmol) was added and the mixture was stirred for one hour. Then N-(3-aminopropyl)-morpholine (140 mg, 0.97 mmol) was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 61% (144 mg, 0.29 mmol). ¹H NMR (CDCl₃, δ: 0.86-0.90 (3H, t, CH); 1.08 (3H, s, CH); 1.29-1.32 (4H, m, CH); 1.41 (3H, s, CH); 1.56-1.59 (2H, m, CH); 1.64-1.70 (1H, m, CH); 1.66 (3H, s, CH); 1.76-1.79 (2H, m, CH), 1.88-1.91 (1H, m, CH); 2.13-2.16 (2H, m, CH); 2.42 (4H, bs, CH); 2.45-2.49 (2H, t, CH); 2.56-2.77 (2H, m, CH); 3.19-3.22 (1H, d, CH); 3.44-3.50 (2H, m, CH); 3.59 (4H, bs, CH); 6.20 (1H, s, CH); 6.39 (1 h, bs, CH); 7.00 (1H, bs, NH). ¹³C NMR (CDCl₃), δ: 14.11, 19.47, 22.57, 23.47, 25.11, 27.59, 31.12, 31.35, 32.01, 33.81, 35.10, 39.62, 45.03, 45.81, 57.94, 66.58, 76.84, 77.16, 78.20, 110.14, 110.44, 110.98, 123.97, 133.78, 139.32, 156.69, 159.52, 171.08. Molecular ion observed [M-H]⁺=485 consistent with the molecular formula C₂₉H₄₄N₂O₃.

### Example 10. Synthesis of (6aR,0aR)-1-hydroxy-6,6,9-trimethyl-N-(2-morpholinoethyl)-3-pentyl-6a,7,8,10a-tetrahydro-6H-benzo[c]chromene-2-carboxamide (Ia₁₀)

To a solution of THCA (170 mg, 0.47 mmol) in dry THF (5 ml) CDI (173 mg, 1.07 mmol) was added and the mixture was stirred for one hour. Then 2-morpholinoethan-1-amine (157 mg, 1.2 mmol) in 5 ml THF was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 47% (106 mg, 0.22 mmol). ¹H NMR (CDCl₃), δ: 0.87-0.93 (3H, t, CH); 1.10 (3H, s, CH); 1.26-1.35 (5H, m, CH); 1.43 (3H, s, CH); 1.62-1.1.70 (3H, m, CH); 1.67 (3H, s, CH); 1.92-1.94 (1H, m, CH); 2.18-2.19 (2H, m, CH); 2.55 (4H, bs, CH); 2.63-2.67 (2H, t. CH); 2.2.69-2.73 (2H, q, CH); 3.23-3.25 (1H, d, CH); 3.58-3.60 (2H, t, CH); 3.75 (4H, bs, CH); 6.24 (1H, s, CH); 6.43 (1 h, s, CH); 6.55 (1H, bs, NH). Molecular ion observed [M-H]⁺=471 consistent with the molecular formula C₂₈H₄₂N₂O₄.

### Example 11. Synthesis of 1-hydroxy-6,6,9-trimethyl-3-pentyl-N-(pyridin-4-ylmethyl)-6a,7,8,10a-tetrahydro-6H-benzo[c]chromene-2-carboxamide (Ia₁₁)

To a solution of THCA (68 mg, 0.19 mmol) in dry THF (5 ml) CDI (55 mg, 0.34 mmol) was added and the mixture was stirred for one hour. Then pyridin-4-ylmethanamine (147 mg, 0.43 mmol) in 5 ml DMF was added and the reaction mixture was stirred overnight. Water was added and the mixture was extracted twice with ether and washed with brine. After drying over sodium sulfate, the solvent was evaporated and crude oil was purified by column chromatography (silica, PA-EA). Yield 31% (28 mg, 0.06 mmol). ¹H NMR (CD₃OD), δ: 0.85-0.89 (3H, t, CH); 1.06 (3H, s, CH); 1.21-1.32 (9H, m, CH); 1.40 (3H, s, CH); 1.46-1.51 (3H, m, CH), 1.57-1.62 (2H, m, CH); 1.67 (3H, s, CH); 1.95-1.98 (1H, m, CH); 2.17-2.19 (2H, m, CH); 2.457-2.59 (2H, t, CH); 3.23-3.25 (2H, bd, CH); 3.64-3.78 (8H, m, CH); 4.54-4.65 (2H, dt, CH); 6.21 (1H, s, CH); 6.33 (1 h, s, CH); 7.50-7.52 (2H, d, CH); 8.48-8.50 (2H, d, CH). ¹³C NMR (CD₃OD), δ: 14.36, 19.23, 23.50, 26.16, 27.83, 28.11, 30.90, 32.15, 32.91, 34.58, 34.94, 35.39, 47.46, 78.51, 111.54, 111.86, 124.41, 125.11, 126.13, 134.53, 142.12, 150.06, 152.86, 156.42, Molecular ion observed [M-H]⁺=449 consistent with the molecular formula C₂₈H₃₆N₂O₃.

### Example 12. Synthesis of N-(cyclopropylmethyl)-4-fluoro-1-hydroxy-6,6,9-trimethyl-3-pentyl-6H-benzo[c]chromene-2-carboxamide (Ia₁₂)

To a solution of compound Ia₅ (48 mg, 0.11 mmol) in dry CH₂C₁₂ (2 ml) 1-fluoropyridinium tetrafluoroborate (32 mg, 0.19 mmol) was added and the mixture was stirred for seventeen hours. 0.5 N HCl was added and the mixture was extracted twice with CH₂Cl₂ and washed with brine. After drying over sodium sulfate, the solvent was evaporated, and crude oil was dried under deep vacuum. Product was purified by column chromatography (silica, PE-EA). Yield 19% (7 mg, 0.02 mmol). Molecular ion observed [M-H]⁺=430 consistent with the molecular formula C₂₆H₃₂FNO₃.

### Example 13. Synthesis of (6aR,0aR)-1-hydroxy-6,6,9-trimethyl-3-pentyl-6a,7,8,10a-tetrahydro-6H-benzo[c]chromene-2-carbohydrazide (Ia₁₃)

To a solution of THCA (254 mg, 0.70 mmol) in dry THF (6 ml) CDI (262 mg, 1.61 mmol) was added and the mixture was stirred for one hour. Then hydrazine hydrate (150 mg, 3 mmol) in 1 ml THF was added and the reaction mixture was stirred overnight. Water was added and the mixture was extracted twice with ether and washed with brine. After drying over sodium sulfate, the solvent was evaporated and solid was dried under deep vacuum. Yield 93% (244 mg, 0.65 mmol). ¹H NMR (CDCl₃), δ: 0.87-0.91 (3H, t, CH); 1.08 (3H, s, CH); 1.31-1.45 (9H, m, CH); 1.57-1.70 (6H, m, CH), 1.89-1.92 (1H, m, CH); 2.15-2.17 (2H, m, CH); 2.64-2.68 (2H, t, CH); 3.20.3.23 (1H, bd, CH); 6.22 (1H, s, CH); 6.38 (1 h, s, CH); 7.11 (2H, s, NH); 7.77 (1H, s, NH). ¹³C NMR (CD₃OD), δ: 14.09, 19.53, 22.56, 23.47, 25.14, 27.57, 31.10, 31.34, 31.88, 33.76, 34.84, 45.80, 78.44, 107.53, 110.66, 111.18, 123.81, 133.97, 139.85, 157.39, 160.13, 172.26. Molecular ion observed [M-H]⁺=373 consistent with the molecular formula C₂₂H₃₂N₂O₃.

### Example 14. Synthesis of (6aR,10aR)—N′-acetyl-1-hydroxy-6,6,9-trimethyl-3-pentyl-6a,7,8,10a-tetrahydro-6H-benzo[c]chromene-2-carbohydrazide (Ia₁₄)

Compound Ia₁₃ (244 mg, 0.65 mmol) was dissolved in 3 ml of acetic acid and refluxed for 10 hours. After removing of acid in vacuum, crude solid was purified by column chromatography (silica, hexane-EA). Yield 81% (218 mg, 0.52 mmol). ¹H NMR (CD₃OD), δ: 0.88-0.91 (3H, t, CH); 1.05 (3H, s, CH); 1.31-1.44 (9H, m, CH); 1.56-1.66 (6H, m, CH), 1.94-1.97 (1H, m, CH); 2.08 (3H, s, CH); 2.15-2.17 (2H, m, CH); 2.64-2.68 (2H, t, CH); 3.19-3.22 (1H, bd, CH); 6.20 (1H, s, CH); 6.42 (1 h, s, CH); 7.11 (2H, s, NH); 7.77 (1H, s, NH). ¹³C NMR (CD₃OD), δ: 14.46, 19.39, 20.04, 23.47, 26.21, 27.86, 31.10, 31.79, 32.20, 32.83, 34.12, 35.19, 47.39, 78.55, 111.13, 111.45, 111.55, 125.63, 133.98, 141.59, 156.62, 157.37, 171.18, 172.29. Molecular ion observed [M-H]⁺=415 consistent with the molecular formula C₂₄H₃₄N₂O₄.

### Example 15. Synthesis of N-(adamantan-1-yl)-2-((6aR,0aR)-1-hydroxy-6,6,9-trimethyl-3-pentyl-6a,7,8,10a-tetrahydro-6H-benzo[c]chromene-2-carbonyl)hydrazine-1-carboxamide (Ia₁₅)

Compound Ia₁₃ (80 mg, 0.21 mmol) in 3 ml of toluene and 1-isocyanatoadamantane (62 mg, 0.35 mmol) was added and the mixture was refluxed for 10 hours. After removing of solvent in vacuum, crude solid was purified by column chromatography (silica, hexane-EA). Yield 50% (58 mg, 0.10 mmol). ¹H NMR (CDCl₃), δ: 0.84-0.88 (3H, t, CH); 1.07 (3H, s, CH); 1.24-1.31 (5H, m, CH); 1.42-1.46 (5H, m, CH), 1.57-1.61 (2H, m, CH); 1.68 (3H, s, CH); 1.89-1.92 (2H, m, CH); 1.98-1.99 (7H, m, CH); 2.06-2.09 (4H, m, CH); 2.15-2.17 (6H, t, CH); 2.60-2.71 (2H, m, CH); 3.21-3.24 (1H, bd, CH); 6.26 (1H, s, CH); 6.41 (1H, s, CH); 7.27 (1H, s, NH); 7.65 (1H, s, NH); 10.75 (1H, s, OH). ¹³C NMR (CDCl₃), δ: 13.52, 18.81, 22.02, 22.83, 24.59, 26.98, 28.87, 29.03, 29.20, 30.45, 30.77 m 31.11, 31.20, 33.25, 33.34, 35.27, 35.32, 35.87, 41.42, 44.77, 45.36, 51.30, 76.24, 109.57, 110.15, 110.40, 123.65, 133.09, 140.69, 156.46, 156.59, 157.06, 170.44. Molecular ion observed [M-H]⁺=415 consistent with the molecular formula C₂₄H₃₄N₂O₄.

### Example 16. Synthesis of (6aR,10aR)-1-hydroxy-6,6,9-trimethyl-3-pentyl-N′-((E)-pyrimidin-2-ylmethylene)-6a,7,8,10a-tetrahydro-6H-benzo[c]chromene-2-carbohydrazide (Ia₁₆)

Compound Ia₁₃ (48 mg, 0.13 mmol) was dissolved in 3 ml of ethanol and pyrimidine-2-carbaldehyde (18 mg, 0.16 mmol) was added. The mixture was refluxed for 3 hours. After removing of ethanol in vacuum, crude solid was purified by column chromatography (silica, hexane-EA). Yield 58% (35 mg, 0.07 mmol). ¹H NMR (CD₃OD), δ: 0.85-0.88 (3H, t, CH); 1.10 (3H, s, CH); 1.26-1.32 (9H, m, CH); 1.43 (3H, s, CH); 1.65-1.67 (6H, m, CH), 1.85-1.92 (3H, m, CH); 2.16-2.17 (2H, m, CH); 2.72-2.75 (2H, t, CH); 3.21-3.24 (1H, bd, CH); 6.27 (1H, s, CH); 6.37 (1 h, s, CH); 7.11 (2H, s, NH); 7.27 (1H, t, CH); 8.44 (1H, s, CH); 8.81-8.83 (2H, d, CH); 9.29 (1H, s, NH); 10.68 (1H, s, OH). Molecular ion observed [M-H]⁺=463 consistent with the molecular formula C₂₇H₃₄N₄O₃.

### Example 17. Synthesis of (6aR,0aR)-1-hydroxy-6,6,9-trimethyl-N-(4-morpholinophenyl)-3-pentyl-6a,7,8,10a-tetrahydro-6H-benzo[c]chromene-2-carboxamide (Ia₁₇)

To a solution of THCA (179 mg, 0.5 mmol) in dry THF (5 ml) CDI (189 mg, 1.16 mmol) was added and the mixture was stirred for one hour. Then 4-morpholinoaniline (115 mg, 0.86 mmol) in 5 ml DMF was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 20% (52 mg, 0.10 mmol). ¹H NMR (CD₃OD), δ: 0.83-0.86 (3H, t, CH); 1.07 (3H, s, CH); 1.26-1.33 (9H, m, CH); 1.40 (3H, s, CH); 1.46-1.49 (1H, m, CH), 1.56-1.61 (3H, m, CH); 1.67 (3H, s, CH); 1.95-1.99 (1H, m, CH); 2.16-2.19 (2H, m, CH); 2.62-2.72 (2H, m, CH); 3.12-3.14 (4H, t, CH); 3.3-3.26 (1H, bd, CH); 3.83-3.85 (4H, t, CH); 6.24 (1H, s, CH); 6.35 (1 h, s, CH); 6.96-6.99 (2H, d, CH); 7.52-7.55 (2H, d, CH). ¹³C NMR (CD₃OD), δ: 14.32, 19.35, 23.46, 26.19, 27.84, 31.90, 31.98, 32.21, 33.01, 35.54, 43.35, 47.35, 53.20, 68.00, 111.54, 78.53, 80.69, 88.40, 93.16, 113.08, 117.38, 122.93, 132.98, 140.30, 142.12, 175.82 Molecular ion observed [M-H]⁺=519 consistent with the molecular formula C₃₂H₄₂N₂O₄.

### Example 18. (6aR,0aR)—N-1-dihydroxy-6,6,9-trimethyl-3-pentyl-6a,7,8,10a-tetrahydro-6H-benzo[c]chromene-2-carboxamide (Ia₁₈)

To a solution of THCA (70 mg, 0.19 mmol) in dry THF (5 ml) CDI (82 mg, 0.75 mmol) was added and the mixture was stirred for one hour. Then, o-trimethylsilyl hydroxylamine (150 mg, 1.42 mmol) in 1 ml THF was added and the reaction mixture was stirred overnight. 1N HCl (1 ml) was added and the mixture was stirred for one hour. The mixture was extracted twice with ether and washed with brine. After drying over sodium sulfate, the solvent was evaporated, and crude oil was purified by column chromatography (silica, PE-EA). Yield 43% (29 mg, 0.08 mmol). ¹H NMR (CCl₃D), δ: 0.86-0.88 (3H, t, CH); 1.13 (3H, s, CH); 1.29-1.35 (5H, m, CH); 1.43 (3H, s, CH); 1.54-1.58 (3H, m, CH), 1.73 (3H, s, CH); 2.01-2.03 (1H, m, CH); 2.15-2.18 (2H, m, CH); 2.51-2.55 (2H, m, CH); 3.37-3.39 (1H, m, CH); 4.76 (1H, s, NH); 6.27 (1H, s, CH); 6.47 (1 h, s, CH); 8.24 (1H, s, OH). ¹³C NMR (CCl₃D), δ: 14.16, 20.08, 22.69, 23.51, 24.54, 29.38, 32.94, 33.43, 35.63, 44.61, 45.96, 66.39, 69.41, 82.20, 107.68, 121.64, 123.87, 134.57, 135.50, 147.98, 154.31, 163.29. Molecular ion observed [M-H]⁺=374 consistent with the molecular formula C₂₂H₃₁NO₄.

### Example 19. Synthesis of ((6aR,10aR)-1-hydroxy-6,6,9-trimethyl-3-pentyl-6a,7,8,10a-tetrahydro-6H-benzo[c]chromen-2-yl)(morpholino)methanone (Ia₁₉)

To a solution of THCA (136 mg, 0.37 mmol) in dry THF (5 ml) CDI (134 mg, 0.82 mmol) was added and the mixture was stirred for one hour. Then morpholine (142 mg, 1.63 mmol) in 1 ml THF was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 43% (68 mg, 0.16 mmol). ¹H NMR (CD₃OD), δ: 0.89-0.93 (3H, t, CH); 1.06 (3H, s, CH); 1.31-1.39 (9H, m, CH); 1.42 (3H, s, CH); 1.54-1.58 (4H, m, CH), 1.66 (3H, s, CH); 1.94-1.99 (1H, m, CH); 2.17-2.19 (2H, m, CH); 2.42 (2H, bs, CH); 3.25-3.26 (2H, bd, CH); 3.64-3.78 (8H, m, CH); 6.22 (1H, s, CH); 6.26 (1 h, s, CH); ¹³C NMR (CD₃OD), δ: 14.36, 19.26, 23.53, 26.14, 27.82, 31.60, 32.16, 32.94, 33.88, 34.83, 47.49, 64.85, 67.78, 78.32, 111.51, 112.22, 117.66, 124.78, 134.89, 140.37, 156.42, 170.99. Molecular ion observed [M-H]⁺=428 consistent with the molecular formula C₂₆H₃₇NO₄.

### Example 20. Synthesis of (6aR,10aR)-6,6,9-trimethyl-2-(((3-morpholinopropyl) amino)methyl)-3-pentyl-6a,7,8,10a-tetrahydro-6H-benzo[c]chromen-1-ol (Ia₂₁)

To a solution of 2 ml LiAlH₄ (2 mmol) in 5 ml of THF compound Ia₉ (80 mg, 0.16 mmol) in 2 ml of THF was added and the mixture was refluxed for 8 hours. After cooling of reaction mixture with ice, 2 ml of brine was added and the mixture was stirred for one hour until white precipitate was obtained. The mixture was filtrated and filtrate was evaporated. Crude oil was purified by column chromatography (silica, hexane-EA). Yield of product 31% (24 mg, 0.05 mmol). ¹H NMR (CD₃OD), δ: 0.88-0.92 (3H, t, CH); 1.05 (3H, s, CH); 1.29-1.39 (6H, m, CH); 1.40 (3H, s, CH); 1.55-1.60 (4H, m, CH); 1.66 (3H, s, CH); 1.80-1.83 (1H, m, CH); 1.85-1.89 (2H, m, CH); 1.94-1.97 (1H, m, CH); 2.15-2.22 (2H, m, CH); (2 2.48-2.50 (4H, m, CH); 2.58-2.63 (2H, m, CH); 3.20-3.23 (1H, d, CH); 3.41-3.44 (1H, t, CH); 3.56-3.57 (2H, m, CH); 3.64-3.67 (3H, m, CH); 3.72-3-76 (2H, m, CH); 6.07 (1H, s, CH); 6.12 (1 h, s, CH). Molecular ion observed [M-H]⁺=371 consistent with the molecular formula C₂₉H₄₆N₂O₃.

### Example 21. Synthesis of (6aR,0aR)-1-hydroxy-6,6,9-trimethyl-3-pentyl-6a,7,8,10a-tetrahydro-6H-benzo[c]chromene-2-carbonitrile (Ia₂₂)

Compound Ia₁ (89 mg, 0.24 mmol) was dissolved in 5 ml of toluol and phosphorus pentoxide (145 mg, 0.1 mmol) was added. The mixture was refluxed for 3 hours. 1N HCl was added and after stirring for 30 min the organic layer was separated and toluene was evaporated in vacuum. Crude oil was purified by column chromatography (silica, hexane-EA). Yield 33% (27 mg, 0.08 mmol). ¹H NMR (CD₃OH), δ: 0.88-0.92 (3H, t, CH); 1.08 (3H, s, CH); 1.29-1.35 (6H, m, CH); 1.41 (3H, s, CH); 1.53-1.58 (3H, m, CH); 1.64-1.70 (1H, m, CH); 1.65 (3H, s, CH); 1.95-1.98 (1H, m, CH); 2.15-2.18 (2H, m, CH); 2.63-2.78 (2H, t, CH); 3.18-3.21 (1H, d, CH); 6.18 (1H, s, CH); 6.40 (1 h, s, CH); Molecular ion observed [M-H]⁺=341 consistent with the molecular formula C₂₂H₂₉NO₂

### Example 22. Synthesis of (6aR,0aR)-2-(4,5-dihydro-1H-imidazol-2-yl)-6,6,9-trimethyl-3-pentyl-6a,7,8,10a-tetrahydro-6H-benzo[c]chromen-1-ol (Ia₂₃)

Compound Ia₇ (40 mg, 0.1 mmol) in 1 ml of xylene was heated in a sealed tube at 150° C. for one hour. After cooling, the xylene was evaporated and crude solid was purified by column chromatography (silica, hexane-EA). Yield 35% (14 mg, 0.35 mmol) ¹H NMR (CD₃OD), δ: 0.83-0.86 (3H, t, CH); 1.05 (3H, s, CH); 1.32-1.37 (8H, m, CH); 1.39 (3H, s, CH); 1.54-1.62 (4H, m, CH), 1.65 (3H, s, CH); 1.94-1.97 (1H, m, CH); 2.13-2.19 (2H, m, CH); 2.63-2.68 (2H, t, CH); 3.19-3.22 (2H, bd, CH); 3.70-3.74 (4H, m, CH); 6.22 (1H, s, CH); 6.62 (1 h, s, CH); Molecular ion observed [M-H]⁺=383 consistent with the molecular formula C₂₄H₃₄N₂.

### Example 23. Synthesis of (6aR,0aR)-6,6,9-Trimethyl-2-(5-methyl-1,3,4-oxadiazol-2-yl)-3-pentyl-6a,7,8,10a-tetrahydro-6H-benzo[c]chromen-1-ol (Ia₂₄)

Compound Ia₁₄ (218 mg, 0.52 mmol) was dissolved in 5 ml of DCM and SOCl₂ (120 mg, 1 mmol) in 1 ml of DCM was added. The mixture was stirred for 12 hours, then the solvent was evaporated, and crude oil was purified by column chromatography (silica, hexane-EA). Yield 47% (98 mg, 0.24 mmol). ¹H NMR (CD₃OD), δ: 0.88-0.91 (3H, t, CH); 1.06 (3H, s, CH); 1.31-1.44 (9H, m, CH); 1.56-1.66 (6H, m, CH), 1.94-1.97 (1H, m, CH); 2.28 (3H, s, CH); 2.15-2.17 (2H, m, CH); 2.64-2.68 (2H, t, CH); 3.19-3.22 (1H, bd, CH); 6.20 (1H, s, CH); 6.48 (1 h, s, CH); ¹³C NMR (CD₃OD), δ: 14.50, 20.14, 20.85, 23.47, 26.21, 27.86, 31.10, 31.79, 32.20, 32.83, 34.12, 35.19, 47.39, 78.55, 111.13, 111.45, 111.55, 125.63, 133.98, 141.59, 156.62, 157.37, 170.13, 171.18. Molecular ion observed [M-H]⁺=397 consistent with the molecular formula C₂₄H₃₂N₂O₃.

### Example 24. Synthesis of (6aR,0aR)-1-hydroxy-6,6,9-trimethyl-3-pentyl-6a,7,8,10a-tetrahydro-6H-benzo[c]chromene-2-carbothioic S-acid (Ia₂₅)

To a solution of THCA (19 mg, 0.05 mmol) in dry ACN (2 ml) Lawesson's reagent (20 mg, 0.05 mmol) was added and the mixture was refluxed for seven hours. 0.5 N HCl was added and the mixture was extracted twice with CH₂Cl₂ and washed with brine. After drying over sodium sulfate, the solvent was evaporated, and solid was dried under deep vacuum. Crude oil was purified by column chromatography (silica, PE-EA). Yield 21% (4 mg, 0.01 mmol). ¹H NMR (CDCl₃), δ: 0.88-0.90 (3H, t, CH); 1.13 (3H, s, CH); 1.32-1.41 (9H, m, CH); 1.60-1.91 (8H, m, CH); 1.96-2.16 (4H, m, CH); 2.64-2.64 (2H, m, CH); 2.80-2.94 (2H, m, CH); 3.23 (1H, bd, CH); 6.25 (1H, s, CH); 6.411H, s, CH). Molecular ion observed [M-H]⁺=375 consistent with the molecular formula C₂₂H₃₀O₃S.

### Example 25. Synthesis of (6aR,0aR)-1-hydroxy-6,6,9-trimethyl-3-pentyl-N-(2-(pyrimidin-2-ylamino)ethyl)-6a,7,8,10a-tetrahydro-6H-benzo[c]chromene-2-carboxamide (Ia₃₁)

To a solution of THCA (56 mg, 0.15 mmol) in dry THF (5 ml) CDI (21 mg, 0.19 mmol) was added and the mixture was stirred for one hour. Then N¹-(pyrimidin-2-yl)ethane-1,2-diamine in 1 ml THF (27 mg, 0.2 mmol) was added and the reaction mixture was stirred overnight. Water was added and the mixture was extracted twice with ether and washed with brine. After drying over sodium sulfate, the solvent was evaporated, and crude oil was purified by column chromatography (silica, PE-EA). Yield 31% (15 mg, 0.31 mmol). ¹H NMR (CD₃OD) δ: 0.84-0.86 (3H, t, CH); 1.06 (3H, s, CH); 1.26-1.41 (9H, m, CH); 1.40 (3H, s, CH); 1.65-1.68 (6H, m, CH), 1.85-1.91 (3H, m, CH); 2.16-2.17 (2H, m, CH); 2.72-2.75 (2H, t, CH); 3.21-3.24 (1H, bd, CH); 3.32 (2H, bs, CH); 3.61 (2H, bs, CH); 6.19 (1H, s, CH); 6.34 (1 h, s, CH); 6.62-6.65 (1H, t, CH); 7.00 (1H, s, NH); 8.81-8.27-8.29 (2H, d, CH). Molecular ion observed [M-H]⁺=479 consistent with the molecular formula C₂₈H₃₈N₄O₃.

### Example 26. Synthesis of (6aR,0aR)-1-hydroxy-6,6,9-trimethyl-3-pentyl-N-(2-(pyrrolidin-1-yl)ethyl)-6a,7,8,10a-tetrahydro-6H-benzo[c]chromene-2-carboxamide (Ia₃₂)

To a solution of THCA (48 mg, 0.13 mmol) in dry THF (5 ml) CDI (38 mg, 0.23 mmol) was added and the mixture was stirred for one hour. Then 2-(pyrrolidin-1-yl)ethan-1-amine in 1 ml THF (56 mg, 0.49 mmol) was added and the reaction mixture was stirred overnight. Water was added and the mixture was extracted twice with ether and washed with brine. After drying over sodium sulfate, the solvent was evaporated, and crude oil was purified by column chromatography (silica, PE-EA). Yield 42% (25 mg, 0.05 mmol). ¹H NMR (CD₃OD), δ: 0.87-0.89 (3H, t, CH); 1.08 (3H, s, CH); 1.32-1.33 (4H, m, CH); 1.41 (3H, s, CH); 1.59-1.70 (3H, m, CH); 1.64 (3H, s, CH); 1.88-1.92 (1H, m, CH); 2.14-2.16 (2H, m, CH); 2.41-2.43 (1H, m, CH); 2.58-2.73 (6H, m, CH); 3.48-3.61 (2H, m, CH); 4.07-4.09 (1H, bs, CH); 6.21 (1H, s, CH); 6.29 (H, s, NH), 6.41 (1H, s, CH). Molecular ion observed [M-H]⁺=455 consistent with the molecular formula C₂₈H₄₂N₂O₃.

### Example 27. Synthesis of (6aR,0aR)—N-(2-(1H-imidazol-1-yl)ethyl)-1-hydroxy-6,6,9-trimethyl-3-pentyl-6a,7,8,10a-tetrahydro-6H-benzo[c]chromene-2-carboxamide (Ia₃₃)

To a solution of THCA (79 mg, 0.22 mmol) in dry THF (5 ml) CDI (65 mg, 0.40 mmol) was added and the mixture was stirred for one hour. Then 2-(1H-imidazol-1-yl)ethan-1-amine dihydrochloride (50 mg, 0.27 mmol) in 1 ml THF contained 0.5 ml triethylamine was added and the reaction mixture was stirred overnight. Water was added and the mixture was extracted twice with ether and washed with brine. After drying over sodium sulfate, the solvent was evaporated, and crude oil was purified by column chromatography (silica, PE-EA). Yield 23% (21 mg, 0.05 mmol). ¹H NMR (CDCl₃, δ: 0.87-0.89 (3H, t, CH); 1.08 (3H, s, CH); 1.32-1.33 (4H, m, CH); 1.41 (3H, s, CH); 1.59-1.70 (3H, m, CH); 1.64 (3H, s, CH); 1.88-1.92 (1H, m, CH); 2.14-2.16 (2H, m, CH); 2.26 (6H, s, CH); 2.60-2.73 (2H, m, CH); 3.20-3.23 (1H, m, CH); 3.55-3.68 (2H, t, CH); 4.41-4.55 (2H, t, CH); 6.22 (1H, s, CH); 6.49 (1H, s, CH); 6.74 (1H, d, CH); 7.19 (1H, d, CH); 8.01 (1H, s, CH). Molecular ion observed [M-H]⁺=452 consistent with the molecular formula C₂₇H₃₇N₃O₃.

### Example 28. Synthesis of (8aR,12aR)-3-(2-methoxyethyl)-8,8,11-trimethyl-5-pentyl-8a,9,10,12a-tetrahydro-2H,8H-benzo[3,4]chromeno[6,5-e][1,3]oxazine-2,4(3H)-dione (Ib₁)

To a solution of Ia₄ (48 mg, 0.11 mmol) in dry DCM (5 ml) triphosgene (329 mg, 1.26 mmol) was added and the mixture was stirred for one hour. Then triethylamine (142 mg, 1.4 mmol) was added and the reaction mixture was stirred overnight. Water was added and the mixture was extracted twice with DCM and washed with brine. After drying over sodium sulfate, the solvent was evaporated and crude oil was purified by column chromatography (silica, PE-EA). Yield 49% (24 mg, 0.05 mmol). ¹H NMR (CDCl₃), δ: 0.87-0.91 (3H, t, CH); 1.11 (3H, s, CH); 1.19-1.26 (4H, m, CH); 1.33-1.37 (6H, s, CH); 1.41-1.47 (9H, m, CH); 1.53-1.59 (3H, m, CH); 1.66-1.75 (5H, m, CH); 1.92-1.93 (1H, m, CH); 2.17-2.18 (2H, m, CH); 2.64-2.79 (2H, t, CH); 2.91-2.99 (1H, m, CH); 3.18-3.20 (1H, m, CH); 3.38 (3H, s, CH); 3.67-3.70 (2H, t, CH); 4.27-4.29 (2H, m, CH); 6.21 (1H, s, CH); 6.59 (1 h, s, CH). ¹³C NMR (CDCl₃), δ: 14.18, 19.64, 22.75, 23.55, 25.00, 27.49, 30.48, 32.01, 33.37, 34.38, 40.98, 45.49, 79.50, 111.49, 104.90, 109.93, 117.32, 122.88, 125.17, 125.34, 135.17, 146.41, 148.33, 153.77, 158.42, 160.68. Molecular ion observed [M-H]⁺=442 consistent with the molecular formula C₂₆H₃₅NO₅.

### Example 29. Synthesis of (7aR,11aR)-7,7,10-trimethyl-4-pentyl-7a,8,9,11a-tetrahydro-7H-benzo[3,4]-chromeno[6,5-d]isoxazol-3-ol (Ic₁)

To a solution of THCA (45 mg, 0.12 mmol) in dry THF (3 ml) CDI (53 mg, 0.71 mmol) was added and the mixture was stirred for one hour. Then triethylamine (72.1 mg, 0.71 mmol) in 1 ml THF was added and, in 5 minutes—hydroxylamine hydrochloride (48 mg, 0.65 mmol). Reaction mixture was stirred overnight then water was added and the mixture was extracted twice with ether and washed with brine. After drying over sodium sulfate, the solvent was evaporated and crude oil was purified by column chromatography (silica, PE-EA). Yield of product 34% (15 mg, 0.04 mmol). ¹H NMR (CD₃OD), δ: 0.90-0.94 (3H, t, CH); 1.07 (3H, s, CH); 1.29-1.36 (6H, m, CH); 1.40 (3H, s, CH); 1.47-1.61 (4H, m, CH); 1.66 (3H, s, CH); 1.93-1.97 (1H, m, CH); 2.15-2.17 (2H, m, CH); 2.75-2.80 (2H, t, CH); 3.18-3.21 (1H, d, CH); 6.16 (1H, s, CH); 6.37 (1 h, s, CH); 6.37 (1H, bs, NH). ¹³C NMR (CD₃OD), δ: 14.43, 19.63, 23.53, 23.59, 26.18, 27.84, 30.90, 32.20, 32.79, 33.23, 34.87, 37.67, 47.36, 79.47, 104.83, 112.68, 113.05, 125.17, 134.21, 146.45, 159.92, 164.55. Molecular ion observed [M-H]⁺=356 consistent with the molecular formula C₂₂H₂₉NO₃.

### Example 30. Synthesis of (1′R,2′R)—N-(2-(dimethylamino)ethyl)-2,6-dihydroxy-5′-methyl-4-pentyl-2′-(prop-1-en-2-yl)-1′,2′,3′,4′-tetrahydro-[1,1′-biphenyl]-3-carboxamide (Ia₂₆)

To a solution of CBDA (41 mg, 0.11 mmol) in dry THF (3 ml) CDI (38 mg, 0.23 mmol) was added and the mixture was stirred for three hours. Then N,N-dimethylethanediamine (50 mg, 0.56 mmol) in 1 ml THF was added and the reaction mixture was stirred overnight. Water was added and the mixture was extracted twice with ether and washed with brine. After drying over sodium sulfate, the solvent was evaporated and crude oil was purified by column chromatography (silica, PE-EA). Yield 67% (32 mg, 0.07 mmol). ¹H NMR (CD₃OD), δ: 0.88-0.91 (3H, t, CH); 0.98-1.00 (6H, d, CH); 1.25-1.34 (5H, m, CH); 1.60-1.1.73 (4H, m, CH); 1.78 (3H, s, CH); 1.93-2.00 (1H, m, CH); 2.07-2.11 (1H, bd, CH); 2.18-2.22 (1H, m, CH); 2.37-2.43 (1H, m, CH); 3.22-3.34 (2H, m, CH); 4.06-4.08 (1H, bs, CH); 4.40 (1H, bs, CH); 4.53 (1H, bs, CH); 5.55 (1H, s, CH); 6.22 (1H, s, CH); 6.30 (H, s, NH); 11.23 (1H, s OH). Molecular ion observed [M-H]⁺=429 consistent with the molecular formula C₂₆H₄₀N₂O₃.

### Example 31. Synthesis of (1′R,2′R)-2,6-dihydroxy-N-isobutyl-5′-methyl-4-pentyl-2′-(prop-1-en-2-yl)-1′,2′,3′,4′-tetrahydro-[1,1′-biphenyl]-3-carboxamide (Ia₂₇)

To a solution of CBDA (33 mg, 0.09 mmol) in dry THF (3 ml) CDI (38 mg, 0.23 mmol) was added and the mixture was stirred for three hours. Then 2-methylethylamine (100 mg, 1.36 mmol) in 2 ml DMF was added and the reaction mixture was stirred overnight. Water was added and the mixture was extracted twice with ether and washed with brine. After drying over sodium sulfate, the solvent was evaporated, and crude oil was purified by column chromatography (silica, PE-EA). Yield 56% (21 mg, 0.05 mmol). ¹H NMR (CD₃OD), δ: 0.88-0.91 (3H, t, CH); 0.98-1.00 (6H, d, CH); 1.27-1.34 (6H, m, CH); 1.58-1.65 (2H, m, CH); 1.70 (3H, s, CH); 1.78-1.81 (3H, m, CH); 1.85-1.93 (1H, m, CH); 2.07-2.11 (1H, bd, CH); 2.18-2.22 (1H, m, CH); 2.37-2.43 (1H, m, CH); 3.22-3.34 (2H, m, CH); 4.06-4.08 (1H, bs, CH); 4.40 (1H, bs, CH); 4.53 (1H, bs, CH); 5.55 (1H, s, CH); 6.22 (1H, s, CH); 6.30 (H, s, NH); 11.23 (1H, s OH). Molecular ion observed [M-H]⁺=414 consistent with the molecular formula C₂H₃₉NO₃.

### Example 32. Synthesis of (1′R,2′R)-2,6-dihydroxy-5′-methyl-4-pentyl-2′-(prop-1-en-2-yl)-N-(2-(pyrrolidin-1-yl)ethyl)-1′,2′,3′,4′-tetrahydro-[1,1′-biphenyl]-3-carboxamide (Ia₂₈)

To a solution of CBDA (48 mg, 0.0.13 mmol) in dry THF (5 ml) CDI (38 mg, 0.23 mmol) was added and the mixture was stirred for three hours. Then 2-(pyrrolidin-1-yl)ethan-1-amine (100 mg, 0.87 mmol) in 1 ml THF was added and the reaction mixture was stirred overnight. Water was added and the mixture was extracted twice with ether and washed with brine. After drying over sodium sulfate, the solvent was evaporated, and crude oil was purified by column chromatography (silica, PE-EA). Yield 32% (34 mg, 0.07 mmol ¹H NMR (CD₃OD), δ: 0.87-0.90 (3H, t, CH); 1.27-1.32 (5H, m, CH); 1.58-1.63 (2H, m, CH); 1.70 (3H, s, CH); 1.76-1.82 (7H, m, CH); 2.06-2.16 (1H, bd, CH); 2.19-2.27 (1H, m, CH); 2.41-2.43 (1H, m, CH); 2.58-2.73 (6H, m, CH); 3.48-3.61 (2H, m, CH); 4.07-4.09 (1H, bs, CH); 4.41 (1H, bs, CH); 4.52-4.53 (1H, bs, CH); 5.55 (1H, s, CH); 6.21 (1H, s, CH); 6.29 (H, s, NH) Molecular ion observed [M-H]⁺=455 consistent with the molecular formula C₂₈H₄₂N₂O₃.

### Example 33. Synthesis of (1′R,2′R)-2,6-dihydroxy-5′-methyl-N-(3-morpholinopropyl)-4-pentyl-2′-(prop-1-en-2-yl)-1′,2′,3′,4′-tetrahydro-[1,1′-biphenyl]-3-carboxamide (Ia₂₉)

To a solution of CBDA (177 mg, 0.49 mmol) in dry THF (5 ml) CDI (162 mg, 1.02 mmol) was added and the mixture was stirred for three hours. Then N-(3-aminopropyl)-morpholine (240 mg, 1.66 mmol) in 1 ml THF was added and the reaction mixture was stirred overnight. Water was added and the mixture was extracted twice with ether and washed with brine. After drying over sodium sulfate, the solvent was evaporated, and crude oil was purified by column chromatography (silica, PE-EA). Yield 49% (120 mg, 0.24 mmol). ¹H NMR (CD₃OD), δ: 0.88-0.91 (3H, t, CH); 1.19 (3H, s, CH); 1.27-1.31 (6H, m, CH); 1.51-1.64 (3H, s, CH); 1.54-1.58 (4H, m, CH), 1.64-1.66 (4H, m, CH); 1.71 (3H, s, CH); 1.75-1.81 (5H, m, CH); 2.02-2.06 (1H, bd, CH); 2.46-2.58 (9H, m, CH); 3.38-3.41 (2H, t, CH); 3.75-3.94 (5H, t, CH); 3.98-4.00 (1H, bd, CH); 4.45-4-47 (2H, d, CH), 5.32 (1H, s, CH); 6.20 (1H, s, CH). Molecular ion observed [M-H]⁺=485 consistent with the molecular formula C₂₉H₄₄N₂O₄.

### Example 34. Synthesis of 2,6-dihydroxy-5′-methyl-4-pentyl-2′-(prop-1-en-2-yl)-1′,2′,3′,4′-tetrahydro-[1,1′-biphenyl]-3-carbohydrazide (Ia₃₀)

To a solution of CBDA (48 mg, 0.12 mmol) in dry THF (3 ml) CDI (38 mg, 0.23 mmol) was added and the mixture was stirred for one hour. Then hydrazine hydrate (30 mg, 0.5 mmol) in 1 ml THF was added and the reaction mixture was stirred overnight. Water was added and the mixture was extracted twice with ether and washed with brine. After drying over sodium sulfate, the solvent was evaporated and solid was dried under deep vacuum. Crude oil was purified by column chromatography (silica, PE-EA). Yield 41% (21 mg, 0.05 mmol). ¹H NMR (CDCl₃), δ: 0.84-0.88 (3H, t, CH); 1.32-1.37 (4H, m, CH); 1.40-1.43 (1H, m, CH); 1.50-1.63 (4H, m, CH); 1.79 (3H, s, CH); 2.06-2.12 (2H, m, CH); 2.65-2.69 (2H, m, CH); 3.95-3.97); 4.11 (2H, bs, CH); 5.48 (1H, s, CH); 6.24 (1H, s, CH); 6.45 (1H, bs, NH); 7.05 (1H, s, NH); 10.85 (1H, bs, OH). Molecular ion observed [M-H]⁺=373 consistent with the molecular formula C₂₂H₃₂N₂O₃.

### Example 35. Study of Anti-Cancer Activity (Chempartner, China)

In this study, the biological activity of certain compounds of the present invention on cancer cell cytotoxicity/anti-proliferation panel was tested using the CellTiter-Glo® cell viability assay. The compounds were dissolved in DMSO to make 10 mM solution.

The cell lines tested were all selected from the Chempartner's cell collection and included: Caco-2 ATCC colon adenocarcinoma; TE-6 RIKEN esophagus, carcinoma; PC-3 ATCC prostate adenocarcinoma; T47D, breast ductal carcinoma; U251, brain human glioma; A549, lung carcinoma; OMP-2, multiple myeloma; SK-HEP-1, liver adenocarcinoma; PANC-1, pancreas ductal carcinoma; AsPC-1, pancreas, ductal carcinoma; and U-87 MG, brain glioblastoma. Staurosporine was used as the reference compound.

For the assays described herein, absolute inhibition of cells growth (%) and absolute IC₅₀ (Abs IC₅₀; the molar concentration of a substance that reduces the specific binding of a radioligand to 50% of the maximum specific binding) were used to derive a value that can be used to compare results within and across runs in the same assay, as well as between different assays.

The results presented herein indicate the ability of the THCA and CBDA derivatives tested to bind to cancer cells and thereby suppress their growth.

### Materials and Assay Conditions

RPMI1640 (Invitrogen, Cat. No. 11875-093; Lot. No. 1924300)
F-12 K (Invitrogen, Cat. No. 211217-022; Lot. No. 1934968)
MEM (Invitrogen, Cat. No. 11095-080; Lot. No. 1897268)
Fetal bovine serum (FBS; Biological Industries, Cat. No. 04-002-1A, Lot. No. 1609758)
MEM NEAA (Invitrogen, Cat. No. 11140-050; Lot. No. 1846154)
Penicillin-Streptomycin solution (Hyclone, Cat. No. SV30010, Lot. No. J170012)
Glutamax (Gibco, Cat. No. 35050-061; Lot. No. 1919082)
96-well plate, white wall with clear bottom, tissue culture treated (Corning, Cat. No. CLS3903; Lot. No. 31817031)
CellTiter Glo assay kit (Promega, Cat. No. G7573, Lot. No. 0000278149)
Staurosporine (selleck, Cat. No. S1421, Lot. No. #S142105)
DMSO (Sigma, Cat. No. 276855-1L. Lot. No. #STBH2596)

### Experimental Methods

### Cell Seeding

a. Prepare complete medium: Add FBS and appropriate additives according to the information sheet provided by the vendor. Mix gently.
b. Check the cell name and complete medium and passage number marked on the flask. For adherent cell lines, refer to c to k. For suspension cell lines, refer to g to k.
c. Remove and discard culture medium using a vacuum pump.
d. Briefly rinse the cell layer with 0.25% (w/v) trypsin-0.038% (w/v) EDTA solution to remove all traces of serum that contains trypsin inhibitor.
e. Add 3.0 ml of trypsin-EDTA solution to flask and observe cells under an inverted microscope until cell layer is dispersed.
f. Add 8.0 ml of complete growth medium and aspirate cells by gently pipetting.
g. Transfer the cell suspension to a centrifuge tube and centrifuge at 800-1000 rpm for 3-5 minutes.
h. Discard the supernatant using a vacuum pump.
i. Add appropriate volume of complete medium. Suspend the cell pellet by gently pipetting.
j. Count the cell numbers with Vi-cell XR and adjust cells to appropriate density.
k. Add 100 μL of cell suspension to 96-well opaque-walled clear bottom plates according to the planned plate layout and place the plates in the CO₂ incubator overnight.
l. Plate layout:

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | |
|---|---|---|
| Blank | Compound, 10 μM starting, 10-pt, 3-fold dilution | DMSO |
| Blank | | DMSO |
| Blank | Compound, 10 μM starting, 10-pt, 3-fold dilution | DMSO |
| Blank | | DMSO |
| Blank | Compound, 10 μM starting, 10-pt, 3-fold dilution | DMSO |
| Blank | | DMSO |
| Blank | STS, 2 μM starting, 10-pt, 3-fold dilution | DMSO |
| Blank | | DMSO |

Blank = medium only;
DMSO = solvent treated cell control (0.5% DMSO)

### Compound Plate Preparation and Addition:

Plate preparation of testing articles. 10 mM stock solution of each one of the compounds (provided as powder) was prepared in DMSO. The start working solution (2 mM) and 10-point with 3-fold serial dilutions were prepared with DMSO according to the above table.
Staurosporine plate preparation. 0.4 mM staurosporine was prepared in DMSO at working concentration.
Compound addition. 0.5 μL of each of the diluted compounds was transferred to the wells containing 100 μL of the culture medium. The total dilution was 200 fold. Cells were incubated with the compounds for 72 hours at 5% CO₂, 37° C.

### Preparation of Reagents

Thaw the CellTiter-Glo buffer and equilibrate to room temperature prior to use.
Equilibrate the lyophilized CellTiter-Glo substrate to room temperature prior to use.
Transfer the appropriate volume of CellTiter-Glo buffer into the amber bottle containing CellTiter-Glo substrate to reconstitute the lyophilized enzyme/substrate mixture. This forms the CellTiter-Glo reagent.
Mix by gently vortexing, swirling or by inverting the contents to obtain a homogeneous solution. The CellTiter-Glo substrate should go into solution easily in less than one minute.

### Assay Measurement

Observe the cell morphology under an inverted microscope after corresponding treatment.
Equilibrate the plate and its contents to room temperature for approximately 30 minutes.
Add 100 μL of CellTiter-Glo reagent to the assay plate by Multidrop Combi instrument.
Mix contents for 10 minutes on an orbital shaker to induce cell lysis.
Allow the plate to incubate at room temperature for 10 minutes to stabilize luminescent signal.
Paste the clear bottom with white back seal and record luminescence with Enspire. The settings should be: Luminescence, measurement time 0.1 ms.

### Data Analysis

Inhibition (%) and absolute IC₅₀ (μM) of each compound was calculated with XLFit curve fitting software that is compatible to Activity Base. The anticancer activities of the tested compounds were compared with that of THCA, since the anticancer activity of THCA has been studied using both pure THCA (De Petrocellis et al., 2011) and extracts containing additional cannabinoids (Nallathambi et al., 2018).

TABLE 4

| | THCA | | Ia₆ | | Ia₉ | | Ia₁₆ | | STS^{*} | |
|---|---|---|---|---|---|---|---|---|---|---|
| | Inhibition | IC₅₀ | Inhibition | IC₅₀ | Inhibition | IC₅₀ | Inhibition | IC₅₀ | Inhibition | IC₅₀ |
| Cell line | % | μM | % | μM | % | μM | % | μM | % | μM |
| T47D | 18.20 | >10 | 84.19 | 6.09 | 97.90 | 5.52 | 47.20 | >10 | 83.46 | 0.22 |
| U251 | 17.22 | >10 | 70.02 | 7.23 | 68.11 | 8.91 | | | 95.87 | 0.01 |
| A549 | 9.30 | >10 | 66.67 | 7.34 | 77.08 | 5.59 | 70.01 | 5.53 | 93.20 | 0.01 |
| TE-6 | — | — | — | — | 67.27 | 7.36 | | | 100 | 0.01 |
| Caco-2 | — | — | — | — | 60.81 | 8.87 | | | 91.56 | 0.01 |
| OMP-2 | 5.94 | >10 | 47.73 | >10 | 76.40 | 6.72 | | | 100 | 0.01 |
| SK-HEP-1 | 16.52 | >10 | 49.38 | >10 | 78.20 | 5.41 | | | 100 | 0.07 |
| PC-3 | — | — | — | — | 61.13 | 9.94 | | | 78.98 | 0.02 |
| PANC-1 | 15.20 | >10 | 90.10 | 6.02 | 92.19 | 5.46 | 85.71 | 1.88 | 99.83 | 0.04 |
| AsPC-1 | — | — | 54.46 | 9.10 | 64.62 | 7.38 | 67.70 | 4.34 | 88.40 | 0.042 |
| U-87 MG | 10.52 | >10 | 13.79 | >10 | 19.84 | >10 | 73.80 | 3.37 | 94.92 | 0.082 |

*STS—Staurosporine (standard)

TABLE 5

| Compounds | | | | | | |
|---|---|---|---|---|---|---|
| (T47D cells) | Ia₄ | Ia₅ | Ia₁₁ | Ia₁₅ | Ia₂₁ | Ia₂₈ |
| Inhibition % | 3.13 | 7.89 | 15.80 | 14.94 | 21.68 | 14.85 |
| AbsIC₅₀ | >10 | >10 | >10 | >10 | >10 | >10 |

### Example 36. Study of Cancer Activity (Pharma Seed, Israel)

In this study, the cytotoxicity of several compounds of the present invention on lung and pancreatic cancer cell was evaluated.

### Formulations

PANC-1 (pancreatic carcinoma) culture medium: DMEM (High Glucose), supplemented with 10% FBS (heat inactivated), 2 mM L-glutamine, and 1% of Pen/Strep/Amp solution. PANC-1 assay medium: DMEM (High Glucose), supplemented with 1% FBS (heat inactivated), 2 mM L-glutamine, and 1% of Pen/Strep/Amp solution.

A549 (lung carcinoma) culture medium: F12-K, supplemented with 10% FBS (heat inactivated), 2 mM L-glutamine, and 1% of Pen/Strep/Amp solution. A549 assay medium: F12-K, supplemented with 1% FBS (heat inactivated), 2 mM L-glutamine, and 1% of Pen/Strep/Amp solution.

### Tested Compounds

Each of the tested compounds was dissolved in DMSO to reach a stock concentration of 20 mM (and stored at −15° C.-−25° C. until use. Compound Ia₂₈ (MW 484 g/mol, 3.05 mg total) was dissolved in 0.315 mL; compound Ia₉ (MW 484 g/mol, 2.33 mg total) was dissolved in 0.241 mL; and compound Ia₆ (MW 428.3 g/mol, 2.25 mg total) was dissolved in 0.262 mL.

On the day of incubation with the cells, each compound was diluted in DMSO to reach 2 mM, 1 mM, 0.5 mM, 0.1 mM stock concentrations (1:10, 1:20, 1:40 and 1:200), and each of these stocks was further diluted in assay medium 1:100 for final concentration of 20 μM, 10 μM, 5 μM, and 1 μM.

### Experimental Methods

Part 1—cell number calibration. PANC-1 and A549 cells were each plated in a 96 well plate, in their culture medium, at various cell concentrations per well, in triplicates, and were allowed to attach for 24 hours. Thereafter, culture medium was discarded, assay medium was added, and the cells were incubated for additional 72 hours. Finally, assay medium was discarded, and fresh culture medium was added to the cells along with 50 μl XTT reagent. The OD was measured every two hours for 6 hours (three times).

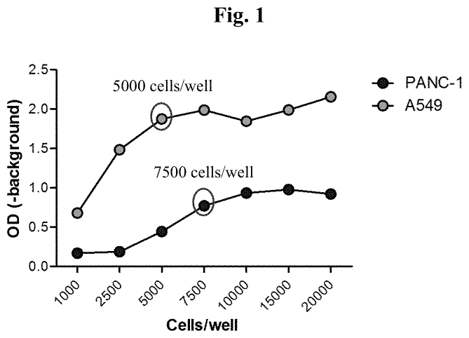

As shown in FIG. 1, after two hours incubation with XTT (after 4 and 6 hours with XTT the OD was too high, exceeding OD of 2), the linear cell growth of PANC-1 cells before reaching plateau level was between 2,500 and 7,500 cells/well, while the linear cell growth of A549 before reaching plateau level was between 1,000 and 5,000 cells/well. The highest cells number/well were chosen for part 2 as follows: PANC-1 at 7,500 cells per well, A549 at 5000 cells per well.

Part 2—cells incubation with tested compounds. PANC-1 and A549 cells were plated in a 96 well plate, in their culture medium, at the optimal cell concentration according to the results from the previous section and were allowed to attach for 24 hours. Thereafter, the culture medium was discarded, and fresh assay medium was added to the cells, supplemented with elevating compound concentrations. Triton 0.1% was used as positive control for cell death. The cells were incubated for another 48 hours (TBD upon microscopic observation). Finally, fresh culture medium was added to the cells along with 50 μl XTT reagent. The OD was measured at the optimal time point according to the results in the previous section.

### Data Analysis

The IC₅₀ data of the compounds Ia₆, Ia₉, Ia₁₆, Ia₂₇, Ia₂₈, Ia₃₁, Ia₃₂, and Ia₃₃ are shown in Table 6.

TABLE 6

| | IC₅₀ (μM) | | | | | | | |
|---|---|---|---|---|---|---|---|---|
| Cell lines | Ia₆ | Ia₉ | Ia₁₆ | Ia₂₇ | Ia₂₈ | Ia₃₁ | Ia₃₂ | Ia₃₃ |
| PANC-1, pancreas, | 9.4 | 11.4 | 7.0 | 7.5 | 19 | 8.5 | 3.9 | 6.7 |
| ductal carcinoma | | | | | | | | |
| A549, lung | 5.2 | 7.8 | — | — | 11 | — | — | — |
| carcinoma | | | | | | | | |

### Conclusion

Both PANC-1 and A549 cell lines exhibited sensitivity to all eight compounds tested. A549 were slightly more sensitive with IC₅₀ range of 5-11 μM, whereas PANC-1's IC₅₀ ranged between 3.9-19 μM. Though PANC-1 and A549 are derived from aggressive and drug resistant pancreases and lung cancers, respectively, all eight compounds tested were found to be potent cytotoxic agents in both cell lines.

### Example 37. Study of Pro- and Anti-Inflammatory Activity Using TNF-α Expression Activation/Deactivation in Peripheral Blood Mononuclear Cells

This study was aimed to test the effect of 12 compounds (THCA, Ia₁, Ia₅, Ia₆, Ia₉, Ia₁₁, Ia₁₅, Ia₁₇, Ia₂₄, Ia₂₅, Ia₂₇, and Ia₂₈) on human peripheral blood mononuclear cells (PBMCs) challenged by lipopolysaccharide (LPS) through monitoring multiple inflammatory endpoints.

Blood samples were collected from healthy volunteers according to protocols that have been reviewed and approved by the Chempartner Institutional Ethic Committee (IEC) upon signing an Informed Consent Form (ICF).

### Methods

Fresh PBMCs were isolated and seeded into 96-well plate, and diluted solutions of the tested compounds and 0.1 μg/mL LPS were then added (in triplicates). To serve as control, 3 wells with PBMCs only, 3 wells with 0.1 μg/mL LPS, and 2 wells with 1 μg/mL dexamethasone (Dex) and 0.1 μg/mL LPS were included. After 72 hours incubation, supernatant was collected for tumor necrosis factor-α (TNF-α) measurement by enzyme-linked immunosorbent assay (ELISA), and cell pellets were harvested for cell proliferation measurement by CellTiter-Glo® Luminescent Cell Viability Assay (CTG).

### Experimental Materials and Methods

VICTOR Nivo Multimode Microplate Reader (PerkinElmer, VICTOR Nivo)
SepMate™-50 (STEMCELL Technologies, Cat. No. A10491-01)
Lymphoprep™ (Stemcell, Cat. No. SV30010, Lot. No. J170012)
Lipopolysaccharides from Escherichia coli 0111:B4 (Sigma, Cat. No L2630-10MG).
Dexamethasone (Alfa Aesar, Cat. No A17590)
Human TNF-α ELISA (Biolegent, Cat. No 430204)
Corning® 96-well Flat Clear Bottom White (Corning, Cat. No 3903)
CellTiter-Glo® Luminescent Cell Viability Assay (Promeda, Cat. No G7572)
Ficoll 400 (CAS 26873-85-8, Merck, Cat. No F2637)

### Experimental Procedure

### PBMCs Isolation

The human blood sample from an individual donor was diluted by the same volume of sterile phosphate buffered solution (PBS) and mixed sufficiently by gentle shake. 15 mL Lymphoprep™ medium was transferred into a new 50 mL centrifuge tube, and the diluted blood sample (30 mL) was then carefully added onto the surface of the medium, which consequently contained Ficoll 400 and blood in a volume ratio of 1:2. Four interfaces were observed after centrifugation, which are, from top to bottom, layers of plasma, mononuclear cells, Ficoll medium, and red blood cells (RBCs). Plasma was absorbed, and mononuclear cells were then transferred into another new sterile centrifuge tube. Then, sterile PBS buffer was added into the collected PBMCs for washing, at a volume ratio of 3:1. PBMCs were washed with 5-10 mL PBS twice before cell counting with cytometer. Cells were centrifuged at 350×g, for 10 min, at 20° C. with the acceleration (5) and deceleration (5) settings during the centrifugation. The cells were resuspended with RPMI 1640 complete medium (containing 10% FBS) for assay, and the density was adjusted with RPMI 1640 complete medium to the final concentration of 2E6 cells/mL. Cells suspension of the PBMCs (100 μL/well) were seeded into 96-well plate (#3903).

### Compounds Solutions Preparation

Diluted solutions of the tested compounds were prepared with RPMI 1640 complete medium according to the following layout. Compounds were prepared at 5-fold of concentration (50 μM), and then were diluted to the working concentration of 10 μM with RPMI 1640 complete medium and made 10-fold serially dilution (2 points). The diluted compounds (50 μL/well) were added to the indicated wells, and the plate was incubated at 37° C., 5% CO₂, for 72 h.

### LPS Induced Cytokine Release Assay

LPS (0.1 μg/mL, 100 μL) was added into each well. Dex (1 μg/mL) was included as a positive control. Plate was incubated at 37° C., 5% CO₂ for 72 h. Supernatant was collected to measure TNF-α by ELISA. Cell proliferation was detected by CTG.

### Conclusions

In all 12 compounds (THCA, Ia₁, Ia₅, Ia₆, Ia₉, Ia₁₁, Ia₁₅, Ia₁₇, Ia₂₄, Ia₂₅, Ia₂₇, and Ia₂₈), 0.1 μg/mL LPS induced obvious increase in the level of TNF-α in human PBMCs. 1 μg/mL Dex efficiently decreased the level of TNF-α by 2.5 folds as compared to LPS-treated cells. According to the data obtained, the compounds tested can be divided into two groups depending on their ability to change the TNF-α expression in the tested concentrations of 1 M and 10 M. The first group includes THCA and its derivatives Ia₅, Ia₆, Ia₉, and Ia₁₇, which increased TNF-α expression in LPS-treated cells; and the second group includes the THCA derivatives Ia₁, Ia₁₁, Ia₁₅, Ia₂₄, and Ia₂₅, as well as the CBDA derivatives Ia₂₇ and Ia₂₈, which decreased TNF-α expression in LPS-treated cells. The ability of Ia₆ and Ia₉ to increase expression of TNF-α together with their inhibition of cell proliferation can explain the antitumor activity of these compounds shown in Examples 35-36. Compound Ia₁ caused a rather pronounced suppression of TNF-α under the tested conditions, which may indicate its potential use as anti-inflammatory drug.

TABLE 7
Data of compounds activities

| | TNF-α pg/ml | | CTG signal 10⁴ | |
|---|---|---|---|---|
| Compound number | 1 μM | 10 μM | 1 μM | 10 μM |
| THCA | 21.04 | 27.57 | 74.89 | 75.54 |
| Ia₁ | 16.30 | 11.85 | 74.10 | 70.03 |
| Ia₅ | 19.60 | 32.33 | 74.73 | 76.26 |
| Ia₆ | 17.09 | 49.73 | 80.67 | 37.67 |
| Ia₉ | 20.26 | 45.82 | 77.55 | 23.31 |
| Ia₁₁ | 28.40 | 15.39 | 72.49 | 74.91 |
| Ia₁₅ | 22.75 | 21.94 | 80.92 | 74.13 |
| Ia₁₇ | 16.44 | 24.59 | 74.83 | 72.61 |
| Ia₂₄ | 32.33 | 28.53 | 77.88 | 62.41 |
| Ia₂₅ | 42.50 | 36.82 | 83.27 | 75.29 |
| Ia₂₇ | 36.23 | 22.09 | 81.90 | 78.67 |
| Ia₂₈ | 41.38 | 22.35 | 76.24 | 72.61 |
| Medium Ctrl + 0.1% DMSO | — | | 109.68 | |
| 0.1 μg/ml LPS + 0.1% DMSO | 18.16 | | 84.50 | |
| 0.1 μg/ml LPS + 1 μg/ml | 5.38 | | 65.51 | |
| Dex + 0.1% DMSO | | | | |

### APPENDIX

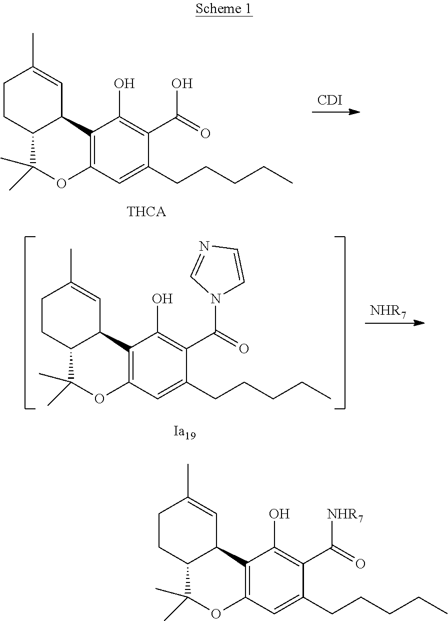

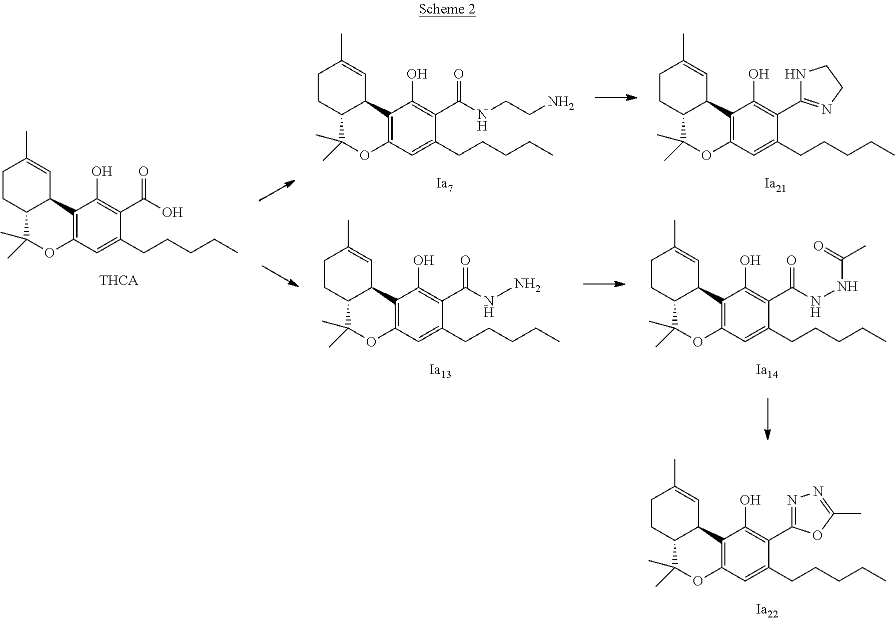 [SMILES: CCCCCc1cc2c(c(O)c1C(=O)O)[C@@H]1C=C(C)CC[C@H]1C(C)(C)O2 ; CCCCCc1cc2c(c(O)c1C(=O)NCCN)[C@@H]1C=C(C)CC[C@H]1C(C)(C)O2 ; CCCCCc1cc2c(c(O)c1C(=O)NN)[C@@H]1C=C(C)CC[C@H]1C(C)(C)O2 ; CCCCCc1cc2c(c(O)c1C(=O)NNC(C)=O)[C@@H]1C=C(C)CC[C@H]1C(C)(C)O2 ; CCCCCc1cc2c(c(O)c1C1=NCCN1)[C@@H]1C=C(C)CC[C@H]1C(C)(C)O2 ; CCCCCc1cc2c(c(O)c1-c1nnc(C)o1)[C@@H]1C=C(C)CC[C@H]1C(C)(C)O2]

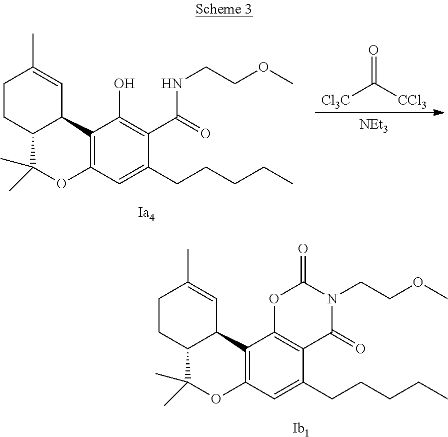

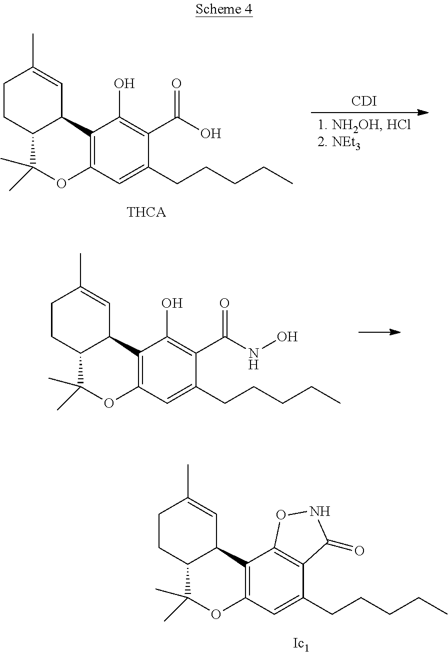

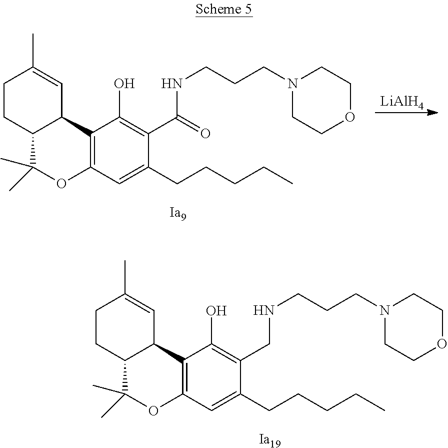

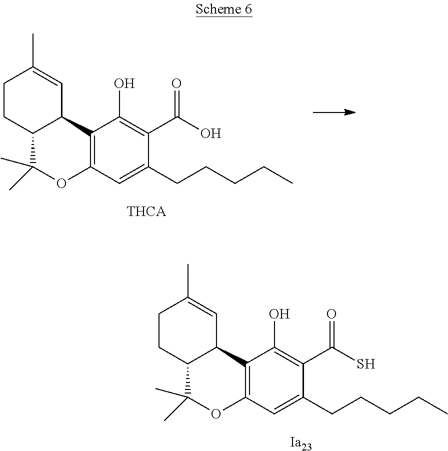

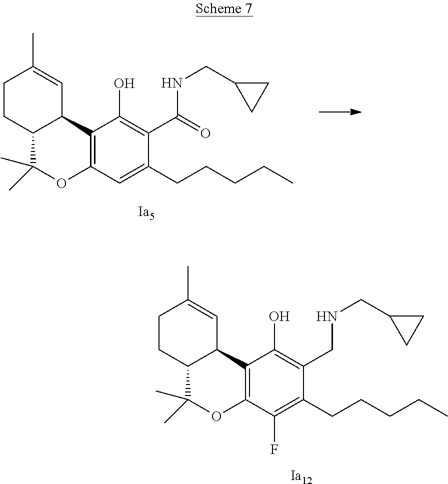

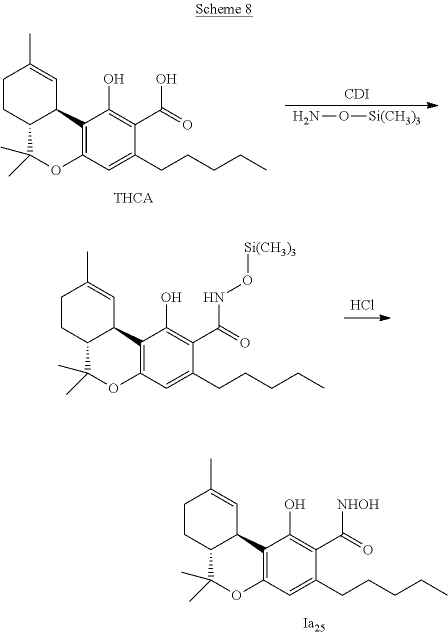

### REFERENCES

Ahmed, S. A.; Ross, S. A.; Slade, D.; RadwASN, m.m.; Zulfiqar, F.; Matsumoto, R. R.; Xu, Y-T.; Virad, E.; Speth, R. C.; Karamyanm V. T.; ElSohly, M. A., Journal of Natural Products, 2008, 71(6), 1119
Crombie, L.; Crombie, W. M. L.; Forbes, R.; Whitaker, A., Journal of Chemical Research, Synopses, 1977, 5, 114-115
Bottaro, J. C.; Bedford, C.; Dodge A., Synthetic Communication, 1985, 15(4), 1333-1335
Chakravarti, B.; Ravi, J.; Ganju, R. K., Oncotarget, 2014, 5, 5852-5872
Crombie, L.; Crombie, W. M. L., Phytochemistry, 1977, 16(9), 1413-1420
Crombie, L.; Crombie, W. M. L.; Forbes, R.; Whitaker, A., Journal of Chemical Research, Synopses, 1977, 5, 114-115
De Petrocellis, L.; Ligresti, A.; Moriello, A. S.; Allara, M.; Bisogno, T.; Petrosino, S.; Stott, C. G.; Di Marzo, V., British J. Pharmacology, 2011, 163(7), 1479-1494
Gaoni, y.; Mechoulam, R., Journal of the American Chemical Society, 1971, 93(1), 217-224
Goto, Y.; Shima, y.; Morimoto, S.; Shoyama, Y.; Murakami, H.; Kusai, A.; Nojima, K., Organic Mass Spectrometry, 1994, 29(11), 668-671
Grotenhermen, F.; Russo, E.; Zuardi, A. W., Cannabis and Cannabinoid Research, 2017, 2 (1),1-4
Harvey, D. J., Biomedical Mass Spectrometry, 1977, 4(2), 88-93
Massi, p.; Valenti, M.; Solinas, M.; Parolaro, D., Cancers, 2010, 2(2), 1013-1026
Moldzio, R.; Pacher, T.; Krewenka, C.; Kranner, B.; Novak, J.; Duvigneau, J. C., Rausch, W. D., Phytomedicine, 2012, 19 (8-9), 819-824
Nallathambi, R.; Mazuz, M.; Namdar, D.; Shik, M.; Namintzer, D.; Vinayaka, A. C.; Ion, A.; Faigenboim, A.; Nasser, A.; Laish, I.; Konikoff, F. M., Koltai, H., Cannabis cannabinoid res., 2018, 3(1), 120-135
Pacher, P., British Journal of Pharmacology, 2013, 168(1), 76-78
Ruhaak, L. R.; Felth, J.; Karlsson, P. C.; Rafter, J. J.; Verpoorte, R.; Bohlin, L., Biol. Pharm. Bull., 2011, 34(5), 774-778
Soderstrom, K.; Soliman, E.; Van Dross, R., Front Pharmacol., 2017, 8, 720
Zehavi, U.; Mechoulam, R., Carbohydrate research, 1981, 98(1), 143-147

What is claimed is:

1. A compound of the formula I: 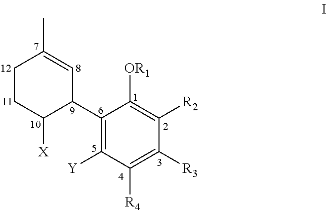
wherein:
X is the radical 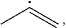
and Y is —OH or —OR₅; or
X is the diradical 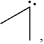
and Y is —O—, and together with X and the carbon atoms to which they are attached form a dihydropyran ring,
or an enantiomer, diastereomer, racemate, or pharmaceutically acceptable salt thereof,
wherein:
R₁ is absent, H, R₅, or —C(O)—;
R₂ is —CN, —C(O)NR₇—, —C(O)N(R₇)₂, —C(O)S—R₇, —C(S)N(R₇)₂, —CH₂N(R₇)₂, or a 5-6-membered aliphatic or aromatic heterocyclyl containing 1-4 heteroatoms, which may be substituted with one or more (C₁-C₄)alkyl groups;
R₃ is —(C₁-C₁₂)alkyl;
R₄ is H, halogen, —NO, —NO₂, or —NH₂;
R₅ is —(C₁-C₁₂)alkyl, —(C₁-C₁₂)haloalkyl, —(C₂-C₁₂)alkenyl, —(C₂-C₁₂)alkynyl, —(C₃-C₁₀)cycloalkyl, —(C₁-C₁₂)alkyl-aryl, —(C₁-C₁₂)alkyl-heterocyclyl, —C(O)—R₆, —SiHₙ(R₆)₃₋ₙ, —BHₙ(R₆)₃₋ₙ, —SO₂—R₆;
R₆ is —(C₁-C₁₂)alkyl, —(C₁-C₁₂)haloalkyl, —(C₆-C₁₂)aryl, or —(C₃-C₁₂)heterocyclyl;
R₇ each independently is H, —OR₈, —N(R₈)₂, —N═C(R₈)₂, —NHC(O)R₈, —NHC(S)R₈, a 5-6-membered aliphatic or aromatic heterocyclic ring containing 1-4 heteroatoms, —(C₁-C₁₂)alkyl substituted with one or more groups each independently selected from the group consisting of —OH, —N(R₉)₂, —O(C₁-C₁₂)alkyl, —(C₃-C₁₀)cycloalkyl, and —(C₃-C₁₂)heterocyclyl, or phenyl which may be substituted with one or more groups each independently selected from the group consisting of —OH, —N(R₉)₂, —O(C₁-C₁₂)alkyl, —(C₃-C₁₀)cycloalkyl, and —(C₃-C₁₂)heterocyclyl, or two R₇ together with the nitrogen atom to which they are attached form a 5- or 6-membered aliphatic or aromatic heterocyclic ring containing 1-4 heteroatoms;
R₈ each independently is H, —(C₁-C₁₂)alkyl, —(C₁-C₁₂)haloalkyl, —(C₃-C₁₀)cycloalkyl, —(C₁-C₁₂)alkyl-aryl, —(C₁-C₁₂)alkyl-heterocyclyl, —(C₆-C₁₂)aryl, —(C₃-C₁₂)heterocyclyl, or —N(R₉)₂;
R₉ each independently is H, —(C₁-C₁₂)alkyl, —(C₁-C₁₂)haloalkyl, —(C₃-C₁₀)cycloalkyl, —(C₁-C₁₂)alkyl-aryl, —(C₁-C₁₂)alkyl-heterocyclyl, —(C₆-C₁₂)aryl, —(C₃-C₁₂)heterocyclyl; and
n is an integer of 0 to 3,
provided that (i) when R₁ is absent or —C(O)—, R₂ is —C(O)NR₇—, and R₁ and R₂ together with the atoms to which they are attached form a 5- or 6-membered heterocyclic ring; and (ii) when R₁ is H or R₅, R₂ is not —C(O)NR₇—; and (iii) when R₂ is —CH₂N(R₇)₂, R are not both H.

2. The compound of claim 1, wherein R₁ is absent, H, or —C(O)—.

3. The compound of claim 1, wherein R₂ is —CN, —C(O)NR₇—, —C(O)N(R₇)₂, —C(O)S—R₇, —CH₂N(R₇)₂, or a 5-6-membered aliphatic or aromatic heterocyclyl containing 1-4 heteroatoms, which may be substituted with one or more (C₁-C₄)alkyl groups.

4. The compound of claim 1, wherein R₃ is —(C₁-C₈)alkyl.

5. The compound of claim 4, wherein R₃ is pentyl.

6. The compound of claim 1, wherein R₄ is H, or halogen.

7. The compound of claim 1, wherein R₅ is —(C₁-C₈)alkyl, or —(C₁-C₈)haloalkyl.

8. The compound of claim 1, wherein:
R₇ each independently is H, —OR₈, —N(R₈)₂, —N═C(R₈)₂, —NHC(O)R₈, —(C₁-C₈)alkyl substituted with one or more groups each independently selected from the group consisting of —OH, —N(R₉)₂, —O(C₁-C₈)alkyl, —(C₃-C₁₀)cycloalkyl, and —(C₃-C₁₂)heterocyclyl, or phenyl which may be substituted with one or more groups each independently selected from the group consisting of —OH, —N(R₉)₂, —O(C₁-C₈)alkyl, —(C₃-C₁₀)cycloalkyl, and —(C₃-C₁₂)heterocyclyl, or two R₇ together with the nitrogen atom to which they are attached form a 5- or 6-membered aliphatic or aromatic heterocyclic ring containing 1-4 heteroatoms; and
R₈ and R₉ each independently is H, —(C₁-C₈)alkyl, —(C₁-C₈)haloalkyl, or —(C₃-C₁₀)cycloalkyl.

9. The compound of claim 1, wherein:
R₁ is absent, H, or —C(O)—;
R₂ is —CN, —C(O)NR₇—, —C(O)N(R₇)₂, —C(O)S—R₇, —CH₂N(R₇)₂, or a 5-6-membered aliphatic or aromatic heterocyclyl containing 1-4 heteroatoms, which may be substituted with one or more (C₁-C₄)alkyl groups;
R₃ is —(C₁-C₈)alkyl;
R₄ is H, or halogen;
R₇ each independently is H, —OR₈, —N(R₈)₂, —N═C(R₈)₂, —NHC(O)R₈, —(C₁-C₈)alkyl substituted with one or more groups each independently selected from the group consisting of —OH, —N(R₉)₂, —O(C₁-C₈)alkyl, —(C₃-C₁₀)cycloalkyl, and —(C₃-C₁₂)heterocyclyl, or phenyl which may be substituted with one or more groups each independently selected from the group consisting of —OH, —N(R₉)₂, —O(C₁-C₈)alkyl, —(C₃-C₁₀)cycloalkyl, and —(C₃-C₁₂)heterocyclyl, or two R₇ together with the nitrogen atom to which they are attached form a 5- or 6-membered aliphatic or aromatic heterocyclic ring containing 1-4 heteroatoms; and
R₈ and R₉ each independently is H, —(C₁-C₈)alkyl, —(C₁-C₈)haloalkyl, or —(C₃-C₁₀)cycloalkyl.

10. The compound of claim 9, wherein R₃ is pentyl.

11. The compound of claim 9, wherein R₁ is H; and R₂ is —CN, —C(O)N(R₇)₂, —C(O)S—R₇, —CH₂N(R₇)₂, or a 5-6-membered aliphatic or aromatic heterocyclyl containing 1-4 heteroatoms, which may be substituted with one or more (C₁-C₄)alkyl groups.

12. The compound of claim 9, wherein R₁ is absent or —C(O)—; R₂ is —C(O)NR₇—, and R₁ and R₂ together with the atoms to which they are attached form a 5- or 6-membered heterocyclic ring.

13. The compound of claim 1, wherein:
X is the diradical 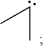
and
Y is —O—, and together with X and the carbon atoms to which they are attached form a dihydropyran ring.

14. The compound of claim 13, wherein:
(i) R₁ is H; R₂ is —C(O)N(R₇)₂; R₃ is pentyl; R₄ is H; and R₇ each is H (herein identified compound Ia₁);
(ii) R₁ is H; R₂ is —C(O)N(R₇)₂; R₃ is pentyl; R₄ is H; one of R₇ is H; and the other one of R₇ is 2-hydroxyethyl (herein identified compound Ia₃);
(iii) R₁ is H; R₂ is —C(O)N(R₇)₂; R₃ is pentyl; R₄ is H; one of R₇ is H; and the other one of R₇ is 2-methoxyethyl (herein identified compound Ia₄);
(iv) R₁ is H; R₂ is —C(O)N(R₇)₂; R₃ is pentyl; R₄ is H; one of R₇ is H; and the other one of R₇ is cyclopropylmethyl (herein identified compound Ia₅);
(v) R₁ is H; R₂ is —C(O)N(R₇)₂; R₃ is pentyl; R₄ is H; one of R₇ is H; the other one of R₇ is 2-(dimethylamino)ethyl (herein identified compound Ia₆);
(vi) R₁ is H; R₂ is —C(O)N(R₇)₂; R₃ is pentyl; R₄ is H; one of R₇ is H; the other one of R₇ is 2-aminoethyl (herein identified compound Ia₇);
(vii) R₁ is H; R₂ is —C(O)N(R₇)₂; R₃ is pentyl; R₄ is H; one of R₇ is H; the other one of R₇ is 2-(dimethylamino)propyl (herein identified compound Ia₈);
(viii) R₁ is H; R₂ is —C(O)N(R₇)₂; R₃ is pentyl; R₄ is H; one of R₇ is H; and the other one of R₇ is 3-(4-morpholinyl)propyl (herein identified compound Ia₉);
(ix) R₁ is H; R₂ is —C(O)N(R₇)₂; R₃ is pentyl; R₄ is H; one of R₇ is H; and the other one of R₇ is 2-(4-morpholinyl)ethyl (herein identified compound Ia₁₀);
(x) R₁ is H; R₂ is —C(O)N(R₇)₂; R₃ is pentyl; R₄ is H; one of R₇ is H; and the other one of R₇ is 4-pyridinylmethyl (herein identified compound Ia₁₁);
(xi) R₁ is H; R₂ is —C(O)N(R₇)₂; R₃ is pentyl; R₄ is F; one of R₇ is H; and the other one of R₇ is cyclopropylmethyl (herein identified compound Ia₁₂);
(xii) R₁ is H; R₂ is —C(O)N(R₇)₂; R₃ is pentyl; R₄ is H; one of R₇ is H; the other one of R₇ is —N(R₈)₂; and R₈ each is H (herein identified compound Ia₁₃);
(xiii) R₁ is H; R₂ is —C(O)N(R₇)₂; R₃ is pentyl; R₄ is H; one of R₇ is H; the other one of R₇ is —NHC(O)R₈; and R₈ is methyl (herein identified compound Ia₁₄);
(xiv) R₁ is H; R₂ is —C(O)N(R₇)₂; R₃ is pentyl; R₄ is H; one of R₇ is H; the other one of R₇ is —NHC(O)R₈; R₈ is —N(R₉)₂; one of R₉ is H; and the other one of R₉ is adamantyl (herein identified compound Ia₁₅);
(xv) R₁ is H; R₂ is —C(O)N(R₇)₂; R₃ is pentyl; R₄ is H; one of R₇ is H; the other one of R₇ is —N═C(R₈)₂; one of R₈ is H; and the other one of R₈ is pyrimidin-2-yl (herein identified compound Ia₁₆);
(xvi) R₁ is H; R₂ is —C(O)N(R₇)₂; R₃ is pentyl; R₄ is H; one of R₇ is H; and the other one of R₇ is 4-(4-morpholinyl)phenyl (herein identified compound Ia₁₇);
(xvii) R₁ is H; R₂ is —C(O)N(R₇)₂; R₃ is pentyl; R₄ is H; one of R₇ is H; the other one of R₇ is —OR₈; and R₈ is H (herein identified compound Ia₁₈);
(xviii) R₁ is H; R₂ is —C(O)N(R₇)₂; R₃ is pentyl; R₄ is H; and the two R₇ together with the nitrogen atom to which they are attached form morpholinyl (herein identified compound Ia₁₉);
(xix) R₁ is H; R₂ is —C(O)N(R₇)₂; R₃ is pentyl; R₄ is H; and the two R₇ together with the nitrogen atom to which they are attached form imidazolyl (herein identified compound Ia₂₀);
(xx) R₁ is H; R₂ is —CH₂N(R₇)₂; R₃ is pentyl; R₄ is H; one of R₇ is H; and the other one of R₇ is 3-(4-morpholinyl)propyl (herein identified compound Ia₂₁);
(xxi) R₁ is H; R₂ is —CN; R₃ is pentyl; and R₄ is H (herein identified compound Ia₂₂);
(xxii) R₁ is H; R₂ is 2-imidazoline-2-yl; R₃ is pentyl; R₄ is H (herein identified compound Ia₂₃);
(xxiii) R₁ is H; R₂ is 5-methyl-1,3,4-oxadiazol-2-yl; R₃ is pentyl; R₄ is H (herein identified compound Ia₂₄);
(xxiv) R₁ is H; R₂ is —C(O)SR₇; R₃ is pentyl; R₄ is H; and R₇ is H (herein identified compound Ia₂₅);
(xxv) R₁ is H; R₂ is —C(O)N(R₇)₂; R₃ is pentyl; R₄ is H; one of R₇ is H; and the other one of R₇ is —CH₂—CH₂—N(R₉)₂; one of R₉ is H; and the other one of R₉ is pyrimidin-2-yl (herein identified compound Ia₃₁);
(xxvi) R₁ is H; R₂ is —C(O)N(R₇)₂; R₃ is pentyl; R₄ is H; one of R₇ is H; and the other one of R₇ is 2-(pyrrolidine-1-yl)ethyl (herein identified compound Ia₃₂);
(xxvii) R₁ is H; R₂ is —C(O)N(R₇)₂; R₃ is pentyl; R₄ is H; one of R₇ is H; and the other one of R₇ is 2-(1H-imidazol-1-yl)ethyl (herein identified compound Ia₃₃);
(xxviii) R₁ is —C(O)—; R₂ is —C(O)NR₇—; R₃ is pentyl; R₄ is H; and R₇ is 2-methoxyethyl (herein identified compound Ib₁); or
(xxix) R₁ is absent; R₂ is —C(O)NR₇—; R₃ is pentyl; R₄ is H; and R₇ is H (herein identified compound Ic₁).

15. The compound of claim 1, wherein:
X is the radical 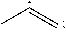
Y is —OH or —OR₅; and
R₅ is —(C₁-C₈)alkyl, or —(C₁-C₈)haloalkyl.

16. The compound of claim 15, wherein Y is —OH.

17. The compound of claim 16, wherein:
(i) R₁ is H; R₂ is —C(O)N(R₇)₂; R₃ is pentyl; R₄ is H; one of R₇ is H; and the other one of R₇ is 2-(dimethylamino)ethyl (herein identified compound Ia₂₆);
(ii) R₁ is H; R₂ is —C(O)N(R₇)₂; R₃ is pentyl; R₄ is H; one of R₇ is H; and the other one of R₇ is 2-(pyrrolidine-1-yl)ethyl (herein identified compound Ia₂₇);
(iii) R₁ is H; R₂ is —C(O)N(R₇)₂; R₃ is pentyl; R₄ is H; one of R₇ is H; and the other one of R₇ is 3-(morpholin-4-yl)propyl (herein identified compound Ia₂₈); or
(iv) R₁ is H; R₂ is —C(O)N(R₇)₂; R₃ is pentyl; R₄ is H; one of R₇ is H; the other one of R₇ is —N(R₈)₂; and R₈ each is H (herein identified compound Ia₃₀).

18. A pharmaceutical composition comprising a compound according to claim 1, or an enantiomer, diastereomer, racemate, or pharmaceutically acceptable salt thereof, and a pharmaceutically acceptable carrier.

19. The pharmaceutical composition of claim 18, comprising a compound selected from the group consisting of the herein identified compounds Ia₁, Ia₃₋₂₈, and Ia₃₀₋₃₃, Ib₁, and Ic₁ in Tables 2-3, or an enantiomer, diastereomer, racemate, or pharmaceutically acceptable salt thereof.

20. A method for CB1 and/or CB2 receptor activation/deactivation in a subject in need thereof, comprising administering to said subject a therapeutically effective amount of a compound according to claim 1, or an enantiomer, diastereomer, racemate, or pharmaceutically acceptable salt thereof.

21. The method of claim 21, wherein said subject suffers from a cancer, inflammatory disease, pain or a condition associated therewith, brain or spinal cord disease, skin disease, immunological disease including autoimmune disease, neurologic disease, neurodegenerative disease or disorder, or neuroinflammatory condition.

22. The method of claim 22, wherein said cancer is adenocarcinoma including colon adenocarcinoma, prostate adenocarcinoma, and liver adenocarcinoma; carcinoma including esophagus carcinoma, pancreas ductal carcinoma, breast ductal carcinoma, and lung carcinoma; multiple myeloma; brain glioma, or brain glioblastoma.
